(19) United States
(12) Patent Application Publication
(10) Pub. No.: US 2024/0206711 A1
(43) Pub. Date: Jun. 27, 2024

(54) SYSTEM AND METHOD TO CONTROL CAMERA RELATIVE TO INSTRUMENTS IN ROBOTIC SYSTEM

(71) Applicant: Auris Health, Inc., Redwood City, CA (US)

(72) Inventors: Charles J. Scheib, Loveland, OH (US); Clinton Denlinger, Cincinnati, OH (US); Jeffrey S. Swayze, West Chester, OH (US); Samuel J. Malanowski, Portland, OR (US); Theodore Leclere, San Francisco, CA (US); Erica D. Chin, Mountain View, CA (US)

(21) Appl. No.: 18/390,295

(22) Filed: Dec. 20, 2023

Related U.S. Application Data
(60) Provisional application No. 63/434,281, filed on Dec. 21, 2022.

(51) Int. Cl. A61B 1/00; A61B 1/313; A61B 34/30

(52) U.S. Cl. CPC A61B 1/00149; A61B 1/00078; A61B 1/00154; A61B 34/30; A61B 1/3132; A61B 2034/301

(57) ABSTRACT

An apparatus includes a scope base configured to be positioned extracorporeally relative to a patient. The scope base is configured to attach to at least one drive mechanism. The apparatus also includes an outer sheath extending distally from the scope base, an insertion channel extending through each of the scope base and the outer sheath, and a scope shaft actuatable relative to the scope base and slidably disposed within the insertion channel. The scope shaft includes a rigid proximal shaft portion configured to be driven by the at least one drive mechanism. The scope shaft also includes a deflectable distal shaft portion. The deflectable distal shaft portion is deflectable relative to the proximal shaft portion. The scope shaft further includes a distal end configured to provide visualization of a body cavity.

### PRIORITY

This application claims the benefit of U.S. Pat. App. No. 63/434,281, entitled “System and Method to Control Camera Relative to Instruments in Robotic System,” filed Dec. 21, 2022, the disclosure of which is incorporated by reference herein.

### BACKGROUND

A variety of surgical instruments include an end effector for use in conventional medical treatments and procedures conducted by a medical professional operator, as well as applications in robotically assisted surgeries. Such surgical instruments may be directly gripped and manipulated by a surgeon or incorporated into robotically assisted surgery. In the case of robotically assisted surgery, the surgeon may operate a master controller to remotely control the motion of such surgical instruments at a surgical site. The controller may be separated from the patient by a significant distance (e.g., across the operating room, in a different room, or in a completely different building than the patient). Alternatively, a controller may be positioned quite near the patient in the operating room. Regardless, the controller may include one or more hand input devices (such as joysticks, exoskeletal gloves, master manipulators, or the like), which are coupled by a servo mechanism to the surgical instrument. In one example, a servo motor moves a manipulator supporting the surgical instrument based on the surgeon's manipulation of the hand input devices. During the surgery, the surgeon may employ, via a robotic surgical system, a variety of surgical instruments including an ultrasonic blade, a surgical stapler, a tissue grasper, a needle driver, an electrosurgical cautery probe, etc. Each of these structures performs functions for the surgeon, for example, cutting tissue, coagulating tissue, holding or driving a needle, grasping a blood vessel, dissecting tissue, cauterizing tissue, and/or other functions.

While several robotic surgical systems and associated components have been made and used, it is believed that no one prior to the inventors has made or used the invention described in the appended claims.

### BRIEF DESCRIPTION OF THE DRAWINGS

While the specification concludes with claims which particularly point out and distinctly claim this technology, it is believed this technology will be better understood from the following description of certain examples taken in conjunction with the accompanying drawings, in which like reference numerals identify the same elements and in which:

FIG. 1 depicts a perspective view of a first example of a robotic system configured for a laparoscopic procedure;

FIG. 2 depicts a perspective view of a second example of a robotic system;

FIG. 3 depicts an end elevational view of the robotic system of FIG. 2;

FIG. 4 depicts the end elevational view of the robotic system of FIG. 2 including an example of a pair of robotic arms;

FIG. 5 depicts a partially exploded perspective view of the robotic arm of FIG. 4 having an instrument driver and an example of a surgical instrument;

FIG. 6A depicts a side elevational view of the surgical instrument of FIG. 5 in a retracted position;

FIG. 6B depicts a side elevational view the surgical instrument of FIG. 5 in an extended position;

FIG. 7 depicts a schematic view of an example of a surgical scope and a pair of surgical instruments, showing a deflectable shaft portion of the surgical scope and distal ends of the surgical instruments inserted through a body wall and into a body cavity of a patient;

FIG. 8 depicts a perspective view of another example of a robotic system having a robotic arm, a surgical scope having a deflectable distal shaft portion, and a cannula having an articulation feature;

FIG. 9 depicts a schematic side elevational view of the surgical scope and the cannula of FIG. 8;

FIG. 10 depicts a side elevational view of the cannula of FIG. 8, showing the articulation feature in an articulated state;

FIG. 11 depicts a side cross-sectional view of the cannula of FIG. 8 with the articulation feature in an articulated state;

FIG. 12A depicts a perspective view of the surgical scope and the cannula of FIG. 8 inserted into the body cavity of a patient, showing the articulation feature in a first articulated state and the deflectable distal shaft portion in a first deflected state;

FIG. 12B depicts a perspective view of the surgical scope and the cannula of FIG. 12A, showing the articulation feature in a second articulated state and the deflectable distal shaft portion in a second deflected state;

FIG. 12C depicts a perspective view of the surgical scope and the cannula of FIG. 12A, showing the deflectable distal shaft portion inserted further into the body cavity;

FIG. 12D depicts a perspective view of the surgical scope and the cannula of FIG. 12A, showing a distal end of the deflectable distal shaft portion in an articulated state;

FIG. 13 depicts a perspective view of another example of a surgical scope having a rigid proximal shaft portion and a deflectable distal shaft portion constrained by a rigid outer sheath;

FIG. 14A depicts a side cross-sectional view of the surgical scope of FIG. 13, showing a distal tip of the deflectable distal shaft portion in a retracted position;

FIG. 14B depicts a side cross-sectional view of the surgical scope of FIG. 13, showing the distal tip of the deflectable distal shaft portion in an extended position;

FIG. 15 depicts an example of a method for tracking an angle of approach of a scope shaft using an inclinometer of the surgical scope of FIG. 13;

FIG. 16A depicts a side cross-sectional view of another example of a surgical scope having a rigid proximal shaft portion and a deflectable distal shaft portion constrained by a flexible outer sheath, showing a distal tip of the deflectable distal shaft portion in a retracted position;

FIG. 16B depicts a side cross-sectional view of the surgical scope of FIG. 16A, showing the distal tip of the deflectable distal shaft portion in an extended position;

FIG. 17 depicts a perspective view of an example of a robotic system having a surgical scope and a bar-mounted surgical scope grounding feature;

FIG. 18 depicts a perspective view of an example of a robotic system having a surgical scope and a patient-mounted surgical scope grounding feature;

FIG. 19 depicts a top elevational view of an example of a robotic system having a surgical scope with a dual-mounting scope base;

FIG. 20 depicts a front perspective view of the surgical scope of FIG. 19;

FIG. 21 depicts a rear perspective view of the surgical scope of FIG. 19;

FIG. 22A depicts a schematic side view of an example of a surgical scope system having a cannula assembly with a cannula sheath and a surgical scope with a scope shaft and flexible outer sheath that are alongside the cannula sheath, showing a sheath base of the surgical scope in a first position;

FIG. 22B depicts a schematic side view of the surgical scope system of FIG. 22A, showing the sheath base of the surgical scope in a second position;

FIG. 23 depicts a perspective view of a distal portion of the surgical scope system of FIG. 22A, showing the scope shaft passing through a scope port of the cannula assembly, with the outer sheath of the surgical scope omitted;

FIG. 24A depicts a schematic top view of an example of a surgical scope system having a cannula assembly with a proximal sheath and a surgical scope with a scope shaft that is obliquely oriented relative to the proximal sheath, showing a distal tip of the scope shaft in a retracted position;

FIG. 24B depicts a schematic top view of the surgical scope system of FIG. 24A, showing the distal tip of the scope shaft in an extended position;

FIG. 24C depicts a schematic top view of the surgical scope system of FIG. 24A, showing the distal tip of the scope shaft in a cleaning position;

FIG. 25 depicts a perspective view of a distal portion of the surgical scope system of FIG. 24A, showing the scope shaft passing through a scope port of the cannula assembly;

FIG. 26A depicts a schematic side view of an example of a surgical scope system having a cannula assembly with a proximal sheath and a surgical scope with a scope shaft that is coaxial with the proximal sheath, showing a distal tip of the scope shaft in a retracted position;

FIG. 26B depicts a schematic side view of the surgical scope system of FIG. 26A, showing the distal tip of the scope shaft in an extended position;

FIG. 26C depicts a schematic side view of the surgical scope system of FIG. 26A, showing the distal tip of the scope shaft in a cleaning position;

FIG. 27 depicts a perspective view of a distal portion of the surgical scope system of FIG. 26A, showing the scope shaft passing through a scope port of the cannula assembly;

FIG. 28A depicts a schematic side view of an example of a surgical scope system having feed rollers, showing a distal tip of the scope shaft in a retracted position; and

FIG. 28B depicts a schematic side view of the surgical scope system of FIG. 28A, showing the distal tip of the scope shaft in an extended position.

The drawings are not intended to be limiting in any way, and it is contemplated that various embodiments of the technology may be carried out in a variety of other ways, including those not necessarily depicted in the drawings. The accompanying drawings incorporated in and forming a part of the specification illustrate several aspects of the present technology, and together with the description serve to explain the principles of the technology; it being understood, however, that this technology is not limited to the precise arrangements shown.

### DETAILED DESCRIPTION

The following description of certain examples of the technology should not be used to limit its scope. Other examples, features, aspects, embodiments, and advantages of the technology will become apparent to those skilled in the art from the following description, which is by way of illustration, one of the best modes contemplated for carrying out the technology. As will be realized, the technology described herein is capable of other different and obvious aspects, all without departing from the technology. Accordingly, the drawings and descriptions should be regarded as illustrative in nature and not restrictive.

It is further understood that any one or more of the teachings, expressions, embodiments, examples, etc. described herein may be combined with any one or more of the other teachings, expressions, embodiments, examples, etc. that are described herein. The following-described teachings, expressions, embodiments, examples, etc. should therefore not be viewed in isolation relative to each other. Various suitable ways in which the teachings herein may be combined will be readily apparent to those of ordinary skill in the art in view of the teachings herein. Such modifications and variations are intended to be included within the scope of the claims.

For clarity of disclosure, the terms “proximal” and “distal” are defined herein relative to a human or robotic operator of the surgical instrument. The term “proximal” refers the position of an element closer to the human or robotic operator of the surgical instrument and further away from the surgical end effector of the surgical instrument. The term “distal” refers to the position of an element closer to the surgical end effector of the surgical instrument and further away from the human or robotic operator of the surgical instrument. It will be further appreciated that, for convenience and clarity, spatial terms such as “side,” “upwardly,” and “downwardly” also are used herein for reference to relative positions and directions. Such terms are used below with reference to views as illustrated for clarity and are not intended to limit the invention described herein.

Aspects of the present examples described herein may be integrated into a robotically-enabled medical system, including as a robotic surgical system, capable of performing a variety of medical procedures, including both minimally invasive, such as laparoscopy, and non-invasive, such as endoscopy, procedures. Among endoscopy procedures, the robotically-enabled medical system may be capable of performing bronchoscopy, ureteroscopy, gastroscopy, etc.

In addition to performing the breadth of procedures, the robotically-enabled medical system may provide additional benefits, such as enhanced imaging and guidance to assist the medical professional. Additionally, the robotically-enabled medical system may provide the medical professional with the ability to perform the procedure from an ergonomic position without the need for awkward arm motions and positions. Still further, the robotically-enabled medical system may provide the medical professional with the ability to perform the procedure with improved ease of use such that one or more of the instruments of the robotically-enabled medical system may be controlled by a single operator.

### I. EXAMPLE OF ROBOTICALLY-ENABLED MEDICAL SYSTEM

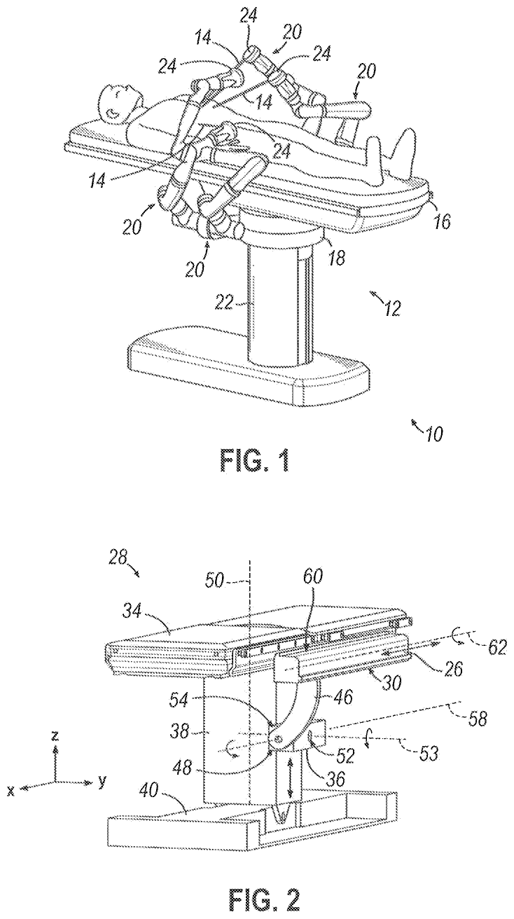

FIG. 1 shows an example of a robotically-enabled medical system, including a first example of a robotic system (10). Robotic system (10) of the present example includes a table system (12) operatively connected to a surgical instrument (14) for a diagnostic and/or therapeutic procedure in the course of treating a patient. Such procedures may include, but are not limited, to bronchoscopy, ureteroscopy, a vascular procedure, and a laparoscopic procedure. To this end, surgical instrument (14) is configured for a laparoscopic procedure, although it will be appreciated that any instrument for treating a patient may be similarly used. At least part of robotic system (10) may be constructed and operable in accordance with at least some of the teachings of any of the various patents, patent application publications, and patent applications that are cited herein.

A. Example of Robotic System with Annular Carriage

As shown in FIG. 1, robotic system (10) includes table system (12) having a platform, such as a table (16), with a plurality of carriages (18) which may also be referred to herein as “arm supports,” respectively supporting the deployment of a plurality of robotic arms (20). Robotic system (10) further includes a support structure, such as a column (22), for supporting table (16) over the floor. Table (16) may also be configured to tilt to a desired angle during use, such as during laparoscopic procedures. Each robotic arm (20) includes an instrument driver (24) configured to removably connect to and manipulate surgical instrument (14) for use. In alternative examples, instrument drivers (24) may be collectively positioned in a linear arrangement to support the instrument extending therebetween along a “virtual rail” that may be repositioned in space by manipulating the one or more robotic arms (20) into one or more angles and/or positions. In practice, a C-arm (not shown) may be positioned over the patient for providing fluoroscopic imaging.

In the present example, column (22) includes carriages (18) arranged in a ring-shaped form to respectively support one or more robotic arms (20) for use. Carriages (18) may translate along column (22) and/or rotate about column (22) as driven by a mechanical motor (not shown) positioned within column (22) in order to provide robotic arms (20) with access to multiples sides of table (16), such as, for example, both sides of the patient. Rotation and translation of carriages (18) allows for alignment of instruments, such as surgical instrument (14), into different access points on the patient. In alternative examples, such as those discussed below in greater detail, robotic system (10) may include a surgical bed with adjustable arm supports including a bar (26) (see FIG. 2) extending alongside. One or more robotic arms (20) may be attached to carriages (18) (e.g., via a shoulder with an elbow joint). Robotic arms (20) are vertically adjustable so as to be stowed compactly beneath table (16), and subsequently raised during use.

Robotic system (10) may also include a tower (not shown) that divides the functionality of robotic system (10) between table (16) and the tower to reduce the form factor and bulk of table (16). To this end, the tower may provide a variety of support functionalities to table (16), such as computing and control capabilities, power, fluidics, optical processing, and/or sensor data processing. The tower may also be movable so as to be positioned away from the patient to improve medical professional access and de-clutter the operating room. The tower may also include a master controller or console that provides both a user interface for operator input, such as keyboard and/or pendant, as well as a display screen, including a touchscreen, for pre-operative and intra-operative information, including, but not limited to, real-time imaging, navigation, and tracking information. In some versions, the tower may include gas tanks to be used for insufflation.

B. Example of Robotic System with Bar Carriage

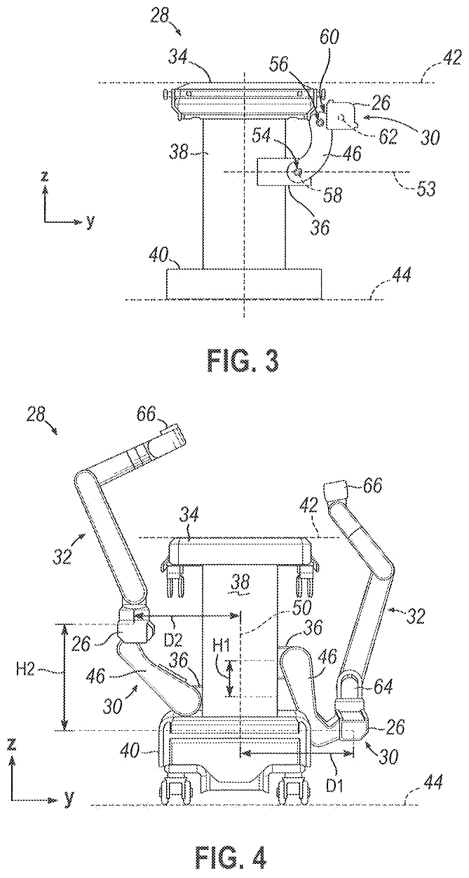

FIGS. 2-4 show another example of a robotic system (28). Robotic system (28) of this example includes one or more adjustable arm supports (30) including bars (26) that are configured to support one or more robotic arms (32) relative to a table (34). In the present example, a single adjustable arm support (30) (FIGS. 2-3) and a pair of adjustable arm supports (30) (FIG. 4) are shown, though additional arm supports (30) may be provided about table (34). Each adjustable arm support (30) is configured to selectively move relative to table (34) so as to alter the position of adjustable arm support (30), and/or any robotic arms (32) mounted thereto, relative to table (34) as desired. Such adjustable arm supports (30) may provide high versatility to robotic system (28), including the ability to easily stow one or more adjustable arm supports (30) with robotic arms (32) beneath table (34).

Each adjustable arm support (30) provides several degrees of freedom, including lift, lateral translation, tilt, etc. In the present example shown in FIGS. 2-4, arm support (30) is configured with four degrees of freedom, which are illustrated with arrows. A first degree of freedom allows adjustable arm support (30) to move in the z-direction (“Z-lift”). For example, adjustable arm support (30) includes a vertical carriage (36). Vertical carriage (36) is configured to move up or down along or relative to a column (38) and a base (40), both of which support table (34). A second degree of freedom allows adjustable arm support (30) to tilt about an axis extending in the y-direction. For example, adjustable arm support (30) includes a rotary joint, which allows adjustable arm support (30) to align with table (34) when table (34) is in a Trendelenburg position or other inclined position. A third degree of freedom allows adjustable arm support (30) to “pivot up” about an axis extending in the x-direction, which may be useful to adjust a distance between a side of table (34) and adjustable arm support (30). A fourth degree of freedom allows translation of adjustable arm support (30) along a longitudinal length of table (34), which extends along the x-direction. Base (40) and column (38) together support table (34) relative to a support surface, which is shown along a support axis (42) above a floor axis (44) in the present example. While the present example shows adjustable arm support (30) mounted to column (38), arm support (30) may alternatively be mounted to table (34) or base (40).

As shown in the present example, adjustable arm support (30) includes vertical carriage (36), a bar connector (46), and bar (26). To this end, vertical carriage (36) attaches to column (38) by a first joint (48), which allows vertical carriage (36) to move relative to column (38) (e.g., such as up and down a first, vertical axis (50) extending in the z-direction). First joint (48) provides the first degree of freedom (“Z-lift”) to adjustable arm support (30). Adjustable arm support (30) further includes a second joint (52), which provides the second degree of freedom (tilt) for adjustable arm support (30) to pivot about a second axis (53) extending in the y-direction. Adjustable arm support (30) also includes a third joint (54), which provides the third degree of freedom (“pivot up”) for adjustable arm support (30) about a third axis (58) extending in the x-direction. Furthermore, an additional joint (56) mechanically constrains third joint (54) to maintain a desired orientation of bar (26) as bar connector (46) rotates about third axis (58). Adjustable arm support (30) includes a fourth joint (60) to provide a fourth degree of freedom (translation) for adjustable arm support (30) along a fourth axis (62) extending in the x-direction.

FIG. 4 shows a version of robotic system (28) with two adjustable arm supports (30) mounted on opposite sides of table (34). A first robotic arm (32) is attached to one such bar (26) of first adjustable arm support (30). This first robotic arm (32) includes a connecting portion (64) attached to a first bar (26). Similarly, a second robotic arm (32) includes connecting portion (64) attached to the other bar (26). As shown in FIG. 4, vertical carriages (36) are separated by a first height (H1), and bar (26) is disposed a second height (H2) from base (40). The first bar (26) is disposed a first distance (D1) from vertical axis (50), and the other bar (26) is disposed a second distance (D2) from vertical axis (50). Distal ends of first and second robotic arms (32) respectively include instrument drivers (66), which are configured to attach to one or more instruments such as those discussed below in greater detail.

In some versions, one or more of robotic arms (32) has seven or more degrees of freedom. In some other versions, one or more robotic arms (32) has eight degrees of freedom, including an insertion axis (1-degree of freedom including insertion), a wrist (3-degrees of freedom including wrist pitch, yaw and roll), an elbow (1-degree of freedom including elbow pitch), a shoulder (2-degrees of freedom including shoulder pitch and yaw), and connecting portion (64) (1-degree of freedom including translation). In some versions, the insertion degree of freedom is provided by robotic arm (32); while in some other versions, an instrument such as surgical instrument includes an instrument-based insertion architecture.

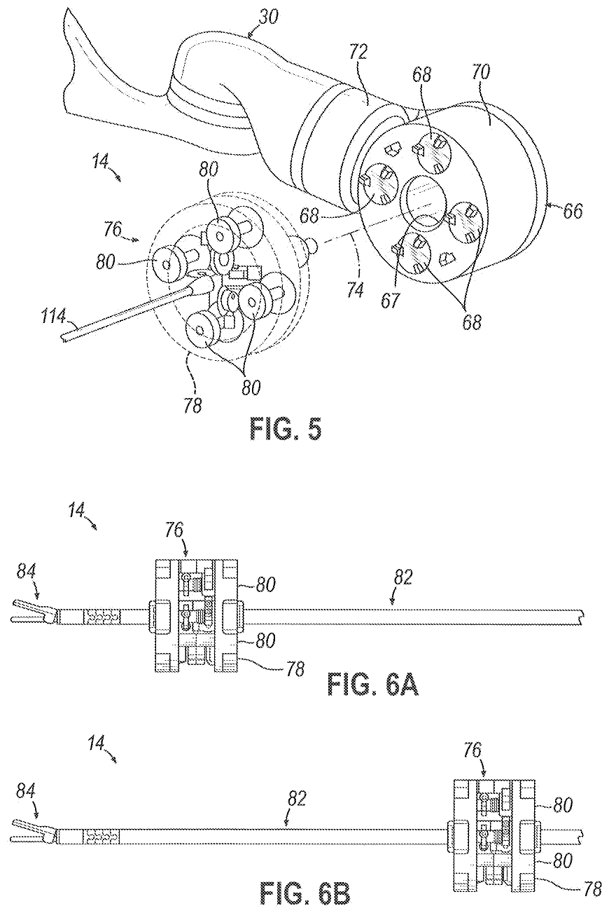

FIG. 5 shows one example of instrument driver (66) in greater detail, with surgical instrument (14) removed therefrom. Given the present instrument-based insertion architecture shown with reference to surgical instrument (14), instrument driver (66) further includes a clearance bore (67) extending entirely therethrough so as to movably receive a portion of surgical instrument (14) as discussed below in greater detail. Instrument driver (66) may also be referred to herein as an “instrument drive mechanism,” an “instrument device manipulator,” or an “advanced device manipulator” (ADM). Instruments may be configured to be detached, removed, and interchanged from instrument driver (66) for individual sterilization or disposal by the medical professional or associated staff. In some scenarios, instrument drivers (66) may be draped for protection and thus may not need to be changed or sterilized.

Each instrument driver (66) operates independently of other instrument drivers (66) and includes a plurality of rotary drive outputs (68), such as four drive outputs (68), also independently driven relative to each other for directing operation of surgical instrument (14). Instrument driver (66) and surgical instrument (14) of the present example are aligned such that the axes of each drive output (68) are parallel to the axis of surgical instrument (14). In use, control circuitry (not shown) receives a control signal, transmits motor signals to desired motors (not shown), compares resulting motor speed as measured by respective encoders (not shown) with desired speeds, and modulates motor signals to generate desired torque at one or more drive outputs (68).

In the present example, instrument driver (66) is circular with respective drive outputs (68) housed in a rotational assembly (70). In response to torque, rotational assembly (70) rotates along a circular bearing (not shown) that connects rotational assembly (70) to a non-rotational portion (72) of instrument driver (66). Power and controls signals may be communicated from non-rotational portion (72) of instrument driver (66) to rotational assembly (70) through electrical contacts therebetween, such as a brushed slip ring connection (not shown). In one example, rotational assembly (70) may be responsive to a separate drive output (not shown) integrated into non-rotatable portion (72), and thus not in parallel to the other drive outputs (68). In any case, rotational assembly (70) allows instrument driver (66) to rotate rotational assembly (70) and drive outputs (68) in conjunction with surgical instrument (14) as a single unit around an instrument driver axis (74).

C. Example of Surgical Instrument with Instrument-Based Insertion Architecture

FIGS. 5-6B show surgical instrument (14) having the instrument-based insertion architecture as discussed above. Surgical instrument (14) includes an elongated shaft assembly (82), an end effector (84) connected to and extending distally from shaft assembly (82), and an instrument base (76) (shown with a transparent external skin for discussion purposes) coupled to shaft assembly (82). Instrument base (76) includes an attachment surface (78) and a plurality of drive inputs (80) (such as receptacles, pulleys, and spools) configured to receive and couple with respective rotary drive outputs (68) of instrument driver (66). Insertion of shaft assembly (82) is grounded at instrument base (76) such that end effector (84) is configured to selectively move longitudinally from a retracted position (FIG. 6A) to an extended position (FIG. 6B), vice versa, and any desired longitudinal position therebetween. As used herein, the retracted position is shown in FIG. 6A and places end effector (84) relatively close and proximally toward instrument base (76); whereas the extended position is shown in FIG. 6B and places end effector (84) relatively far and distally away from instrument base (76). Insertion into and withdrawal of end effector (84) relative to the patient may thus be facilitated by surgical instrument (14), although it will be appreciated that such insertion into and withdrawal may also occur via adjustable arm supports (30) in one or more examples.

When coupled to rotational assembly (70) of instrument driver (66), surgical instrument (14), comprising instrument base (76) and instrument shaft assembly (82), rotates in combination with rotational assembly (70) about the instrument driver axis (74). Since instrument shaft assembly (82) is positioned at the center of instrument base (76), instrument shaft assembly (82) is coaxial with instrument driver axis (74) when attached. Thus, rotation of the rotational assembly (70) causes instrument shaft assembly (82) to rotate about its own longitudinal axis. Moreover, as instrument base (76) rotates with instrument shaft assembly (82), any tendons connected to drive inputs (80) of instrument base (76) are not tangled during rotation. Accordingly, the parallelism of the axes of rotary drive outputs (68), rotary drive inputs (80), and instrument shaft assembly (82) allows for the shaft rotation without tangling any control tendons, and clearance bore (67) provides space for translation of shaft assembly (82) during use.

The foregoing examples of surgical instrument (14) and instrument driver (66) are merely illustrative examples. Robotic arms (32) may interface with different kinds of instruments in any other suitable fashion using any other suitable kinds of interface features. Similarly, different kinds of instruments may be used with robotic arms (32), and such alternative instruments may be configured and operable differently from surgical instrument (14).

In addition to the foregoing, robotic systems (10, 28) may be configured and operable in accordance with at least some of the teachings of U.S. Pat. No. 9,737,371, entitled “Configurable Robotic Surgical System with Virtual Rail and Flexible Endoscope,” issued Aug. 22, 2017, the disclosure of which is incorporated by reference herein, in its entirety; U.S. Pat. No. 10,945,904, entitled “Tilt Mechanisms for Medical Systems and Applications,” issued Mar. 16, 2021, the disclosure of which is incorporated by reference herein, in its entirety; U.S. Pub. No. 2019/0350662, entitled “Controllers for Robotically-Enabled Teleoperated Systems,” published Nov. 21, 2019, the disclosure of which is incorporated by reference herein, in its entirety; U.S. Pub. No. 2020/0085516, entitled “Systems and Methods for Concomitant Medical Procedures,” published Mar. 19, 2020; and/or U.S. Pub. No. 2021/0401527, entitled “Robotic Medical Systems Including User Interfaces with Graphical Representations of User Input Devices,” published Dec. 30, 2021, the disclosure of which is incorporated by reference herein, in its entirety.

### II. EXAMPLE OF A SURGICAL SCOPE HAVING DEFLECTABLE SHAFT

In robotically assisted laparoscopic procedures, it may be desirable to mount a surgical instrument in the form of a surgical scope, also referred to as a surgical camera or a laparoscope, to a robotic arm of the robotic system to provide real-time visualization of a target surgical site and surrounding anatomical structures with the patient's body cavity during the procedure. Some conventional surgical scopes implemented in robotic systems may include an elongate rigid base and an elongate rigid shaft extending distally from the base. The surgical scope may be mounted to the head of a robotic arm, where the head is also docked directly to a cannula through which the scope shaft is inserted to access the body cavity. This configuration may tend to result in the surgical scope and its robotic arm consuming valuable space in the workspace located directly above the patient. This may ultimately tend to restrict the range of motion of the surgical scope and other robotically-controlled surgical instruments operating within this workspace, thereby limiting the reach and access of the surgical scope and the surgical instruments and risking collision between these devices and their respective robotic arms in the workspace. Accordingly, it may be desirable to provide alternative configurations of surgical scopes that promote greater reach and access of the surgical scope and accompanying surgical instruments of a robotic system.

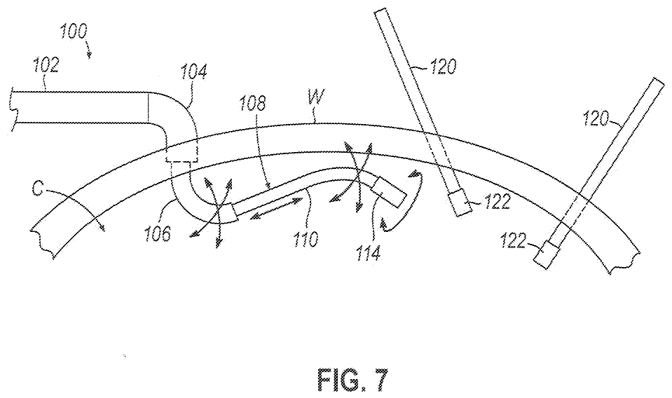

FIG. 7 shows an example of a surgical scope (100) that is constructed such that a portion of surgical scope (100) is non-rigid, thus enabling a base (not shown) of surgical scope (100) and its corresponding robotic arm (not shown) to be positioned remotely from the location at which the surgical scope enters through the body wall (W) and into a body cavity (C) of a patient, such as an abdominal cavity. Surgical scope (100) includes an elongate outer sheath (102) (also referred to as an “outer tube”), which may be rigid or deflectable; and a curved joint (104) at a distal end of outer sheath (102), which may also be rigid with a predefined curvature or deflectable and configured to assume a curved state as shown. As used herein, the term “deflectable” encompasses configurations that are configured to deflect relative to an initial axis, including configurations that are resiliently flexible, malleable, and/or articulatable (e.g., with a plurality of interconnected rigid links), for example. Though not shown, a proximal end of outer sheath (102) is coupled with a scope base that may be mounted to and controlled by a motorized drive mechanism (e.g., similar to instrument driver (66)) of a robotic arm.

Surgical scope (100) further includes a deflectable distal sheath (106) that extends distally from a distal end of outer sheath (102), and a scope shaft (108) that is slidably disposed within outer sheath (102) and distal sheath (106). Scope shaft (108) includes a deflectable distal shaft portion (110) (also referred to as a “leader”) having an articulation section (112) and a distal tip section (114). Distal tip section (114) includes an optical module having a distally facing lens (not shown) configured to provide visualization of a target surgical site within body cavity (C). The deflectable construction of deflectable distal shaft portion (110) enables it to conform to the curvatures of curved joint (104) and distal sheath (106) as scope shaft (108) slidably advances and retracts relative to outer sheath (102) and distal sheath (106). In some versions, scope shaft (108) may further include a rigid proximal shaft portion that is directly connected to deflectable distal shaft portion (110) and that facilitates insertion and retraction of scope shaft (108). Additionally, in some other versions distal sheath (106) may be an independent structure and used with surgical scope (100).

In the present version, distal sheath (106) and scope shaft (108) cooperate to provide surgical scope (100) with six degrees of freedom, as illustrated by respective arrows in FIG. 7. Specifically, first and second degrees of freedom are provided by the ability of distal sheath (106) to articulate in first and second planes that perpendicularly intersect one another, thus providing pitch and yaw at distal sheath (106). By way of example only, one or more pull-wires, drive bands, and/or other actuation members may be used to drive articulation of distal sheath (106). A third degree of freedom is provided by the ability of scope shaft (108) to longitudinally advance and retract relative to distal sheath (106) and outer sheath (102). Fourth and fifth degrees of freedom are provided by the ability of the deflectable distal shaft portion (110) to articulate at articulation section (112) in first and second planes that perpendicularly intersect one another, thus providing pitch and yaw at articulation section (112). By way of example only, one or more pull-wires, drive bands, and/or other actuation members may be used to drive articulation of distal shaft portion (110). A sixth degree of freedom is provided by the ability of distal tip section (114) to rotate about its longitudinal axis relative to a proximal remainder of deflectable distal shaft portion (110), thus providing roll at distal tip section (114). In some other versions, the components of surgical scope (100) may be modified to include more or fewer (e.g., zero) articulation sections, each of which may be configured to articulate in one or more planes, to provide any desired quantity and arrangement of degrees of freedom. By way of example only, in other versions scope shaft (108) may include zero or two or more articulation sections (112), each configured to articulate in one or more intersecting planes.

In use, a surgeon may first create an incision in body wall (W), for example at the umbilicus, to provide access to a target surgical site located within body cavity (C). One or more other surgical instruments (120) may be inserted through body wall (W) at separate locations, for example each with a surgical cannula or other surgical access device. Each surgical instrument (120) may be mounted to a respective robotic arm and includes an end effector (122) (shown schematically) that is operable to grasp tissue, cut tissue, staple tissue, seal tissue, and/or provide other functionality at the target surgical site. Distal sheath (106) of surgical scope (100) is inserted distally through the incision in body wall (W) into body cavity (C) while outer sheath (102) remains supported by a respective robotic arm. Surgical scope (100) may then be actuated, for example by a drive mechanism of the respective robotic arm, to advance scope shaft (108) distally through outer sheath (102) and distal sheath (106) and into body cavity (C), such that distal sheath (106) serves as an introducer cannula. Before, during, or after advancement of scope shaft (108), distal sheath (106) may be articulated by the drive mechanism to a desired articulated state. Additionally, upon exiting distal sheath (106) and entering body cavity (C), articulation section (112) of scope shaft (108) may be driven by the drive mechanism to orient distal tip section (114) in a desired direction. Additionally, distal tip section (114) may be rotated relative to the proximal remainder of scope shaft (108) to provide desired visualization within body cavity (C).

### III. EXAMPLE OF A SURGICAL CANNULA HAVING DISTAL ARTICULATION JOINT

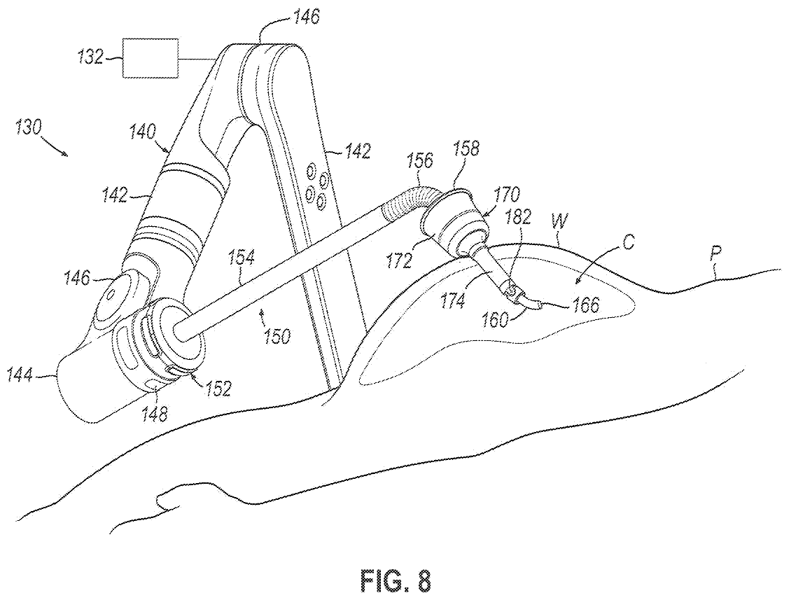

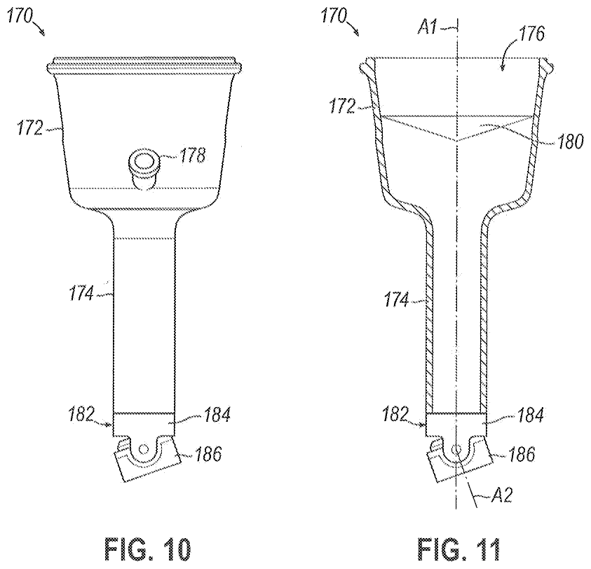

As described above, distal sheath (106) of surgical scope (100) may be configured to articulate in one or more planes to facilitate positioning of distal tip section (114) within body cavity (C) for optimal visualization of a target surgical site and surrounding anatomical structures. In some instances, it may be desirable to combine a deflectable surgical scope with a surgical cannula having a distal articulation feature in the form of an articulation joint. As described below, FIGS. 8-12D show some versions of such surgical cannulas.

FIG. 8 shows an example of a robotic system (130) that includes a robotic arm (140), a surgical instrument in the form of a surgical scope (150) removably coupled to a head (144) of robotic arm (140), and a surgical access device in the form of a cannula (170) coupled with surgical scope (150) remotely from head (144). Robotic arm (140) includes arm segments (142) and head (144) that are interconnected by movable joints (146), where head (144) includes a motorized drive mechanism (148) that may be similar to instrument driver (66) described above. Robotic arm (140) is operable to selectively position and orient surgical scope (150) relative to a patient (P) by driving arm segments (142) and drive mechanism (148) based on control signals received from a master controller (132) of robotic system (130), shown schematically. Master controller (132) may be operatively coupled with robotic arm (140), including surgical scope (150), via a wired connection or a wireless connection, for example.

Robotic arm (140) may be similar in structure and function to any of robotic arms (20, 32) described above, and is mountable to any suitable arm support structure such as a column (22) or any of the other arm support structures disclosed above or in the patent references incorporated by reference herein. Though not shown, robotic system (130) may further include one or more additional robotic arms (140) each supporting and controlling a respective surgical instrument having an end effector of which surgical scope (150) may provide visualization within a body cavity (C) of the patient (P).

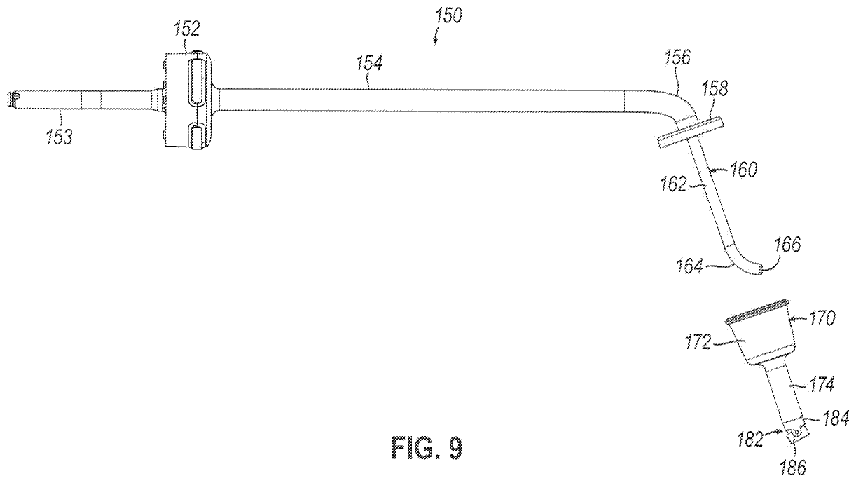

As shown in FIGS. 8 and 9, surgical scope (150) includes a scope base (152), an elongate rigid outer sheath (154) that extends distally from scope base (152), a flexible joint (156) at a distal end of outer sheath (154), a cannula docking plate (158) at a distal end of flexible joint (156), and a scope shaft (160) slidably disposed within outer sheath (154). Flexible joint (156) may have a predefined curved shape or be configured to assume a curved shape as shown and may include an internal sheath (not shown) that resists binding. Similar to scope shaft (108) described above, scope shaft (160) includes a deflectable distal shaft portion (162) that includes an articulation section (164). Scope base (152) may be similar to instrument base (76) described above in that scope base (152) is configured to attach to drive mechanism (148) of robotic arm head (144) such that drive inputs of scope base (152) operatively couple with respective drive outputs of drive mechanism (148). Scope base (152) includes an elongate cylindrical extension (153) (also referred to as a nosecone) that extends coaxially through a central clearance bore (not shown) of drive mechanism (148). Drive mechanism (148) is operable to drive insertion (i.e., longitudinal advancement and retraction) and articulation of scope shaft (160) relative to outer sheath (154). In some versions, drive mechanism (148) may also be further operable to drive articulation of cannula (170), described below.

As shown in FIGS. 8-11, cannula (170) includes a proximal structure in the form of a cup (172) having an open proximal end, and a distal structure in the form of an elongate tube (174) that extends distally from a distal end of cup (172) and has a smaller maximum outer diameter than cup (172). A distal end of cup (172) tapers radially inwardly to the proximal end of tube (174). In the present version, both of cup (172) and tube (174) are rigid such that neither is configured to deflect laterally during use when tube (174) is deployed in a body wall (W). In other versions, all or a portion of tube (174) may be laterally deflectable (e.g., flexible).

As shown in FIG. 11, the interiors of cup (172) and tube (174) cooperate to define a working channel (176) that extends along a central primary axis (A1) of cannula (170) and is sized and configured to receive and guide scope shaft (160) of surgical scope (150) longitudinally therethrough into body cavity (C). A proximal lip of cup (172) is configured to releasably couple with cannula docking plate (158) of surgical scope (150) such that the open proximal end of cup (172) is enclosed by docking plate (158). A distal portion of an annular sidewall of cup (172) includes an access port (178) that communicates with working channel (176). In some versions, access port (178) may be configured to couple with a source of insufflation gas, such as pressurized air, for directing the gas into or out of body cavity (C) to regulate insufflation of body cavity (C). Additionally, or alternatively, access port (178) may be configured to receive various other fluids and/or surgical instruments. Cup (172) further includes an inner seal member (180) (shown schematically) configured to establish an air-tight seal against scope shaft (160) when scope shaft (160) is positioned within working channel (176), and also when scope shaft (160) is removed from working channel (176), to thereby maintain insufflation of body cavity (C) during a procedure. In other versions, cannula (170) and/or surgical scope (150) may include one or more additional seal members configured to maintain insufflation of body cavity (C).

In addition to the foregoing, cannula (170) may be constructed and operable in accordance with at least some of the teachings of U.S. Pat. No. 10,792,069, entitled “Trocar Seal Assemblies,” issued Oct. 6, 2020; U.S. Pat. No. 10,820,924, entitled “Asymmetric Shaft Seal,” issued Nov. 3, 2020; U.S. Pub. No. 2021/0338272, entitled “Pinch to Release Cannula Depth Limiter,” published Nov. 4, 2021; and/or U.S. Pat. No. 10,939,937, entitled “Trocar with Oblique Needle Insertion Portion and Perpendicular Seal Latch,” issued Mar. 9, 2021. The disclosures of these references are incorporated by reference herein, in their entirety.

Cannula (170) further includes an articulation feature in the form of an articulation joint (182) at the distal end of tube (174). Similar to distal sheath (106) of surgical scope (100), articulation joint (182) is configured to facilitate in positioning a distal tip section (166) of surgical scope shaft (160) at a desired location and orientation within body cavity (C) to visualize the target surgical site. Articulation joint (182) of the present example includes a rigid proximal link (184) that is affixed to a distal end of tube (174), and a rigid distal link (186) pivotably coupled with proximal link (184) about a pivot axis that extends transversely to primary axis (A1) of cannula (170). Accordingly, articulation joint (182) of the present version is configured to articulate in a single plane that includes primary axis (A1) to orient distal link (186) along an articulated secondary axis (A2) that is angled relative to primary axis (A1), as shown in FIG. 11. In other versions, distal link (186) may be configured to pivot relative to proximal link (184) about one or more additional pivot axes, and/or articulation joint (182) may include one or more additional links pivotably coupled with proximal and distal links (184, 186) about non-parallel pivot axes, such that articulation joint (182) is configured to articulate in two or more planes that intersect one another and include or extend parallel to primary axis (A1).

Articulation of articulation joint (182) may be active or passive. For instance, articulation joint (182) may be driven by drive mechanism (148) of robotic arm head (144) via one or more articulation drivers (not shown), such as one or more tendons (e.g., pull-wires, drive bands, etc.); or by another drive mechanism positioned remotely from robotic arm head (144), for example as described below. Alternatively, articulation joint (182) may be configured to passively assume an articulated state in response to scope shaft (160) being driven into an articulated state at its articulation section (164). In some such versions, articulation joint (182) may be resiliently biased toward a straight configuration in which distal link (186) is coaxial with primary axis (A1) of cannula (170). In other versions, articulation joint (182) may be manipulated by hand into an articulated state before insertion through body wall (W), and it may be configured to maintain such a preset articulated state. As another variation, another instrument (e.g., grasper) within the cavity (C) may be used to manipulate articulation joint (182) in the patient (P).

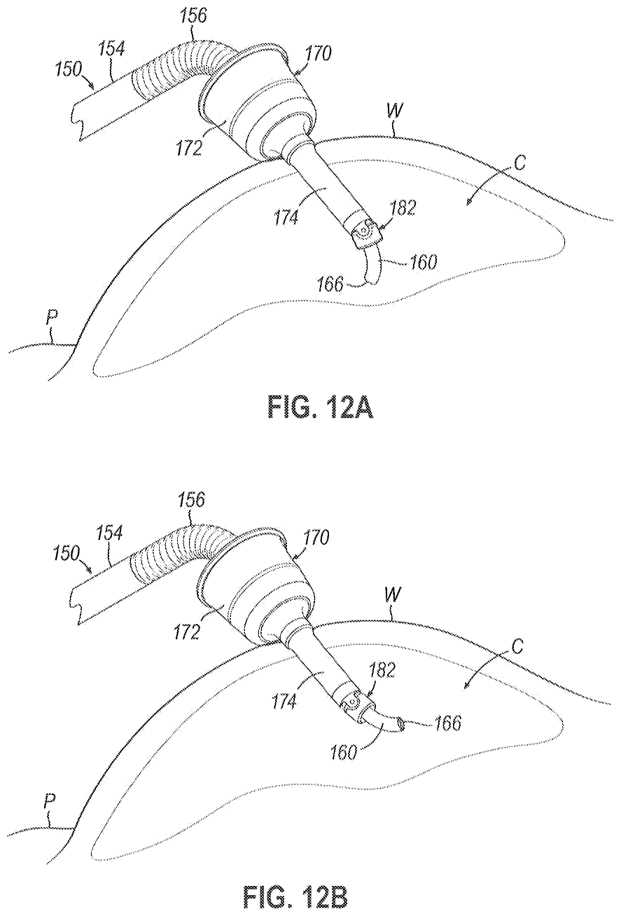

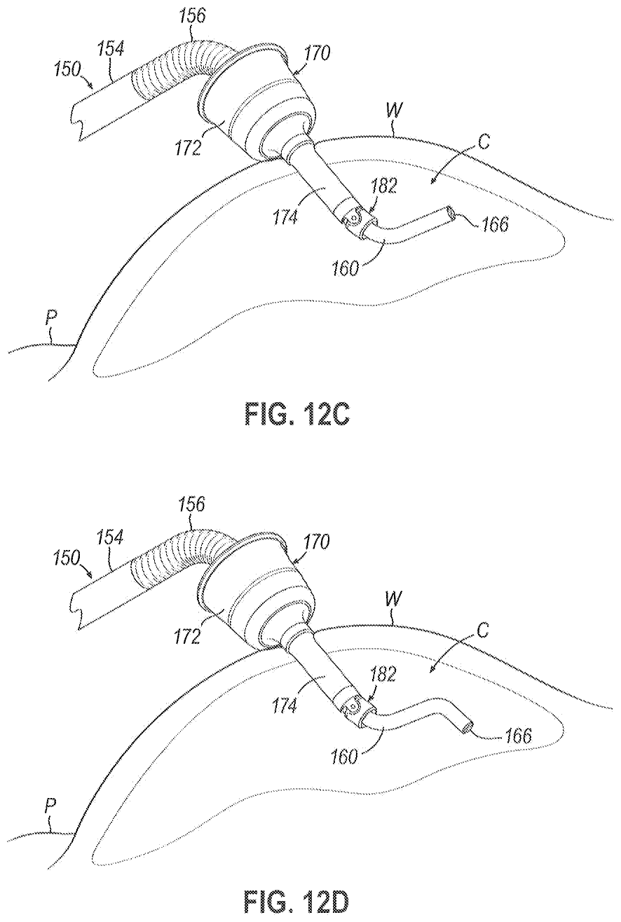

As shown in FIG. 12A, cannula tube (174) is inserted through body wall (W), for example using an obturator (not shown), such that at least the distal end of cannula tube (174) is positioned within body cavity (or “intracorporeally”) and cup (172) is positioned external to body cavity (C) (or “extracorporeally”). In versions where an obturator is used during insertion through body wall (W), the obturator may be removed from cannula tube (174) before surgical scope shaft (160) is inserted through cannula tube (174). As shown in FIG. 8, robotic arm (140) and its head (144) are positioned remotely from cannula (170) such that outer sheath (154) drapes away from cannula (170) via the flexibility of flexible joint (156), thus generally clearing the workspace directly above body wall (W) for use by other robotic arms (not shown) supporting other surgical instruments. In some procedures, cannula (170) may be stabilized relative to the patient with a mechanical grounding feature (not shown), such as a stationary arm. Such a stationary arm may be secured to table (16, 34) or some other grounding structure.

As shown in FIGS. 12A-12D, scope shaft (160) is actuated by drive mechanism (148) distally through cannula (170) and into body cavity (C). FIG. 12A shows articulation joint (182) in a first example of an articulated state in which articulation joint (182) directs scope shaft (160) in a first angled direction within body cavity (C). FIG. 12B shows articulation joint (182) in a second example of an articulated state in which articulation joint (182) directs deflectable scope shaft (160) in a second angled direction within body cavity. As noted above, articulation joint (182) may be configured to transition between such articulated states actively or passively. In cases of active cannula articulation, deflectable distal shaft portion (162) may be flexible along its length to conform to the curvature defined by articulation joint (182) as scope shaft (160) is inserted further into body cavity (C), as shown particularly in FIG. 12C. As shown in FIG. 12D, scope shaft (160) may be articulated at its distal articulation section (164) to suitably orient its distal tip section (166) within body cavity (C) to provide visualization of the target surgical site and/or surrounding anatomical structures. In the example shown in FIG. 12D, scope shaft (160) has achieved a dogleg bend configuration, though scope shaft (160) may alternatively achieve any other suitable kind of articulated state, including but not limited to different articulated states with two or more bends regions at different corresponding positions along the length of scope shaft (160).

### IV. EXAMPLE OF A SURGICAL SCOPE WITH RIGID PROXIMAL SHAFT PORTION AND DEFLECTABLE DISTAL SHAFT PORTION CONSTRAINED BY RIGID OUTER SHEATH

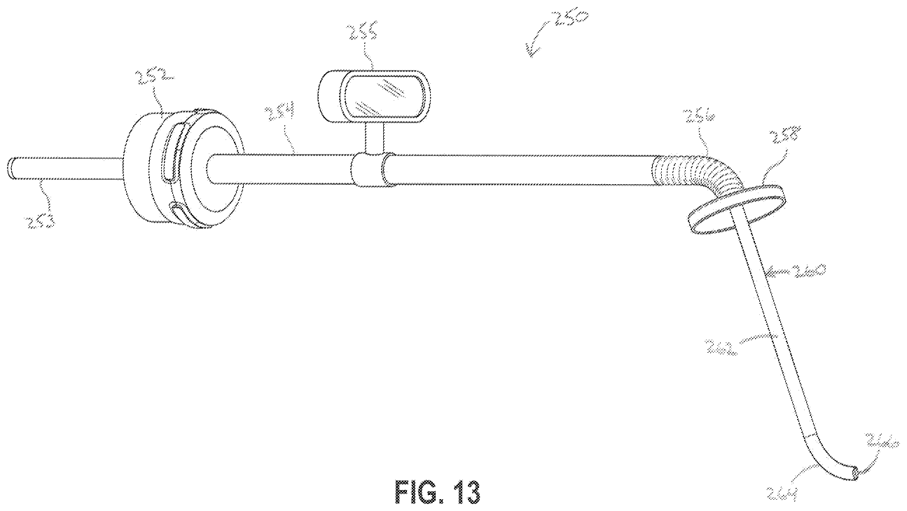

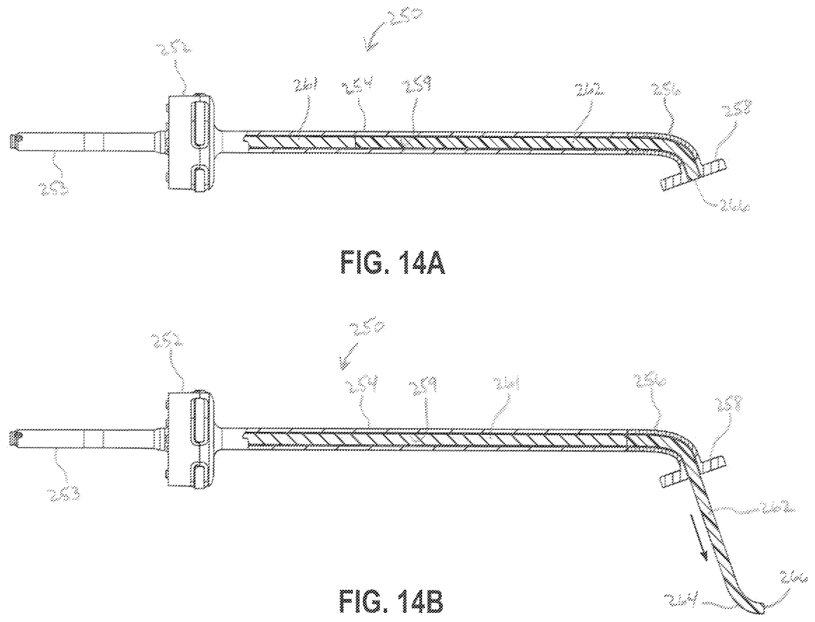

In some instances, it may be desirable to provide a surgical scope that includes a scope shaft having a rigid proximal shaft portion that facilitates insertion and retraction of the scope shaft relative to body cavity (C), while also having a deflectable distal shaft portion that facilitates articulation of the scope shaft to provide visualization of the target surgical site and/or surrounding anatomical structures, and for such a deflectable distal shaft portion to be constrained by a rigid outer sheath. FIGS. 13-14B show an example of an alternative surgical scope (250) that exhibits such functionality. Surgical scope (250) is similar to surgical scope (150) described above, except as otherwise described below. In this regard, surgical scope (250) includes a scope base (252) having a proximal extension (253), an elongate rigid outer sheath (254) that extends distally from scope base (252), an inclinometer (255) secured to outer sheath (254), a flexible joint (256) at a distal end of outer sheath (254), and a cannula docking plate (258) at a distal end of flexible joint (256). As best shown in FIGS. 14A-14B, surgical scope (250) also includes an insertion channel (259) extending through each of scope base (252), outer sheath (254), flexible joint (256), and cannula docking plate (258). Surgical scope (250) of the present example further includes a scope shaft (260) slidably disposed within insertion channel (259). Cannula docking plate (258) may be configured to releasably couple with a surgical access device, such as cannula (170). Flexible joint (256) may have a predefined curved shape or be configured to assume a curved shape as shown and may include an internal sheath (not shown) that resists binding. Scope base (252) is configured to attach to drive mechanism (148) of robotic arm head (144) such that drive inputs of scope base (252) operatively couple with respective drive outputs of drive mechanism (148). Drive mechanism (148) is operable to drive insertion (i.e., longitudinal advancement and retraction) and articulation of scope shaft (260) relative to outer sheath (254).

In the example shown, scope shaft (260) includes a rigid proximal shaft portion (261) and a deflectable distal shaft portion (262). Deflectable distal shaft portion (262) may be similar to deflectable distal shaft portion (162) described above. For example, deflectable distal shaft portion (262) includes an articulation section (264) and a distal tip (266) that includes an optical module having a distally facing lens (not shown) configured to provide visualization of a target surgical site with body cavity (C), such that scope shaft (260) may be articulated at its distal articulation section (264) to suitably orient distal tip (266) within body cavity (C) to provide visualization of the target surgical site and/or surrounding anatomical structures.

Insertion of surgical scope (250) is grounded at scope base (252) such that distal tip (266) is configured to selectively move from a retracted position (FIG. 14A) to an extended position (FIG. 14B), vice versa, and any desired longitudinal position therebetween. In this regard, rigid proximal shaft portion (261) is configured to be selectively driven longitudinally through a straight portion of insertion channel (259) within outer sheath (254). Any suitable features may be used to facilitate the driving of rigid proximal shaft portion (261) by drive mechanism (148), including any one or more of pulleys, cables, carriers, such as a kinetic articulating rotating tool (KART), and/or other structures configured to communicate movement to rigid proximal shaft portion (261). As shown, rigid proximal shaft portion (261) is directly connected to deflectable distal shaft portion (262). In this manner, rigid proximal shaft portion (261) is configured to push deflectable distal shaft portion (262) distally when rigid proximal shaft portion (261) is driven distally by drive mechanism (148), and is configured to pull deflectable distal shaft portion (262) proximally when rigid proximal shaft portion (261) is driven proximally by drive mechanism (148). For example, rigid proximal shaft portion (261) may be configured to push deflectable distal shaft portion (262) distally through a curved portion of insertion channel (259) within flexible joint (256) to thereby extend deflectable distal shaft portion (262) out of insertion channel (259), and may be configured to pull deflectable distal shaft portion (262) proximally back through the curved portion of insertion channel (259) to thereby retract deflectable distal shaft portion (262) into insertion channel (259).

It will be appreciated that deflectable distal shaft portion (262) may be constrained by insertion channel (259) when retracted therein to extend generally coaxially with insertion channel (259), and may be permitted to deflect (e.g., articulate) when extended therefrom. More particularly, deflectable distal shaft portion (262) may be constrained by insertion channel (259) to be generally straight when disposed entirely within the straight portion of insertion channel (259), and to assume a curved shape corresponding to that of flexible joint (256) when disposed at least partially within the curved portion of insertion channel (259). Deflectable distal shaft portion (262) may likewise conform to the curvature defined by articulation joint (182) of cannula (170) as scope shaft (260) is guided into body cavity (C).

As mentioned above, cannula docking plate (258) may be configured to releasably couple with cannula (170), such that cannula (170) may guide deflectable distal shaft portion (262) of scope shaft (260) into body cavity (C) when deflectable distal shaft portion (262) is extended out of insertion channel (259). In some other versions, outer sheath (254) and/or flexible joint (256) may be permanently coupled with cannula (170), such that cannula docking plate (258) may be omitted. For example, cannula (170) may be integrated into the structure of surgical scope (250), such as in a manner similar to that described below.

While outer sheath (254) of the present example is both rigid and straight, outer sheath (254) may have any other suitable configuration. In some versions, outer sheath (254) may be rigid and at least a portion of outer sheath (254) may be curved and/or bent. For example, a distal portion of outer sheath (254) may have a predefined curved and/or bent shape, such that flexible joint (256) may be omitted. Outer sheath (254) may be flexible in other versions. In some versions, outer sheath (254) may include a rigidizing feature such that outer sheath (254) is configured to selectively transition between a flexible state and a rigid state. In still other versions, outer sheath (254) is malleable.

As mentioned above, surgical scope (250) of the present example includes an inclinometer (255) secured to outer sheath (254). Inclinometer (255) is configured to detect an orientation of outer sheath (254) relative to horizontal and to communicate feedback signals indicative of the orientation of outer sheath (254) relative to horizontal to master controller (132) of robotic system (130). As described in greater detail below, such feedback signals may be processed by master controller (132) to determine a position of outer sheath (254), and master controller (132) may operate distal tip (266) responsively to such feedback signals. In the example shown, inclinometer (255) is mounted (e.g., clamped) to an exterior surface of outer sheath (254) at a location between scope base (252) and flexible joint (256), though inclinometer (255) may be secured to outer sheath (254) at any other suitable location and/or in any other suitable manner.

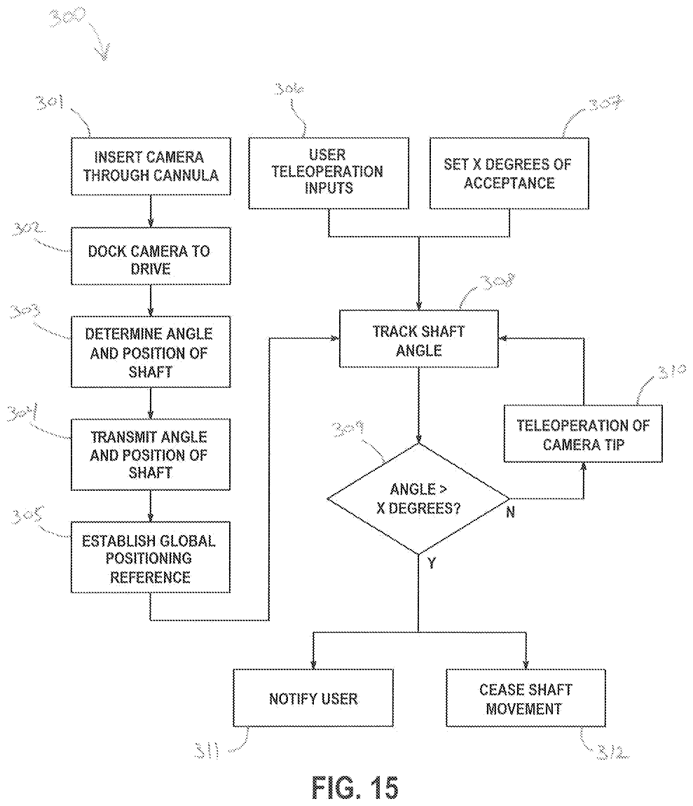

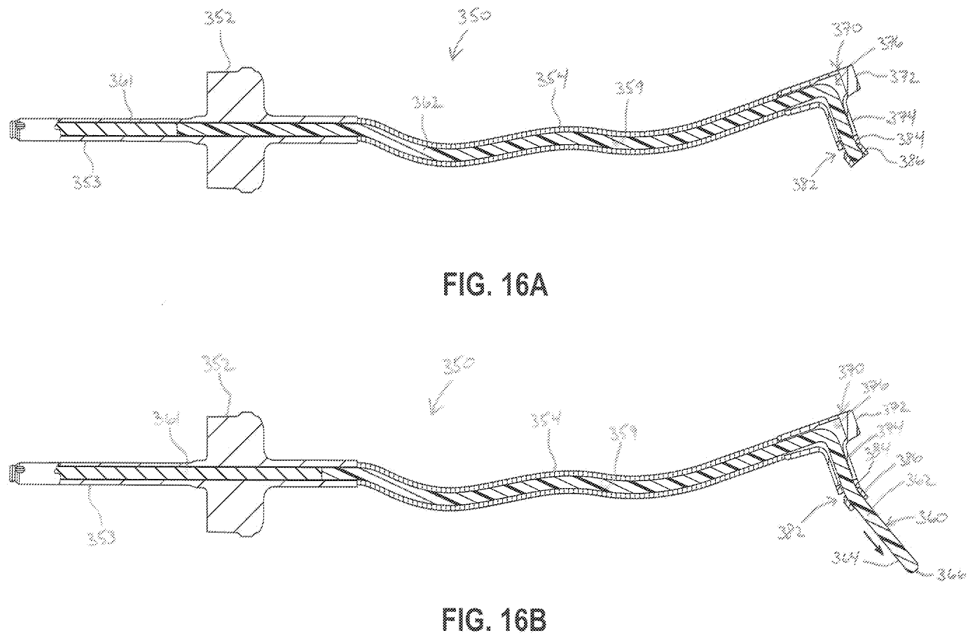

Referring now to FIG. 15, a method (300) for tracking an angle of approach of a scope shaft, such as scope shaft (260), using an inclinometer, such as inclinometer (255) of surgical scope (250), includes step (301), at which scope shaft (260) of surgical scope (250) is at least partially inserted through a cannula, such as cannula (170). Method (300) proceeds from step (301) to step (302), at which scope base (252) of surgical scope (250) is attached to drive mechanism (148) of robotic arm head (144) such that drive inputs of scope base (252) operatively couple with respective drive outputs of drive mechanism (148). Method (300) proceeds from step (302) to step (303), at which an angle and position of scope shaft (260) are determined, such as via inclinometer (255). Method (300) proceeds from step (303) to step (304), at which the angle and position of scope shaft (260) are transmitted, such as via any suitable wireless or wired protocol, to a controller, such as master controller (132). Method (300) proceeds from step (304) to step (305), at which a global positioning reference is established, which may include initial registration of the angle of scope shaft (260) being used in addition to the global position of robotic arm (140). Prior to, after, or in parallel with any one or more of steps (301, 302, 303, 304, 305), method (300) includes step (306), at which user teleoperation inputs are received, as well as step (307), at which a threshold angle of acceptance is set.

Method (300) proceeds from each of steps (305, 306, 307) to step (308), at which the angle of scope shaft (260) is continuously tracked, such as via inclinometer (255), during performance of a surgical operation. Method (300) proceeds from step (308) to step (309), at which a comparison between the tracked angle of scope shaft (260) and the threshold angle of acceptance is performed.

If a determination is made at step (309) that the tracked angle is not greater than the threshold angle of acceptance, then method (300) proceeds from step (309) to step (310), at which teleoperation of distal tip (266) of scope shaft (260) is performed. Method (300) returns from step (310) to step (308) for continued tracking of the angle of scope shaft (260).

If a determination is made at step (309) that the tracked angle is greater than the threshold angle of acceptance, then method (300) proceeds from step (309) to step (311), at which a notification is provided to the user that the tracked angle is greater than the threshold angle of acceptance, and/or to step (312), at which movement of scope shaft (260) is ceased.

It will be appreciated that other position verification techniques may be used in addition to or in lieu of tracking the orientation of outer sheath (254). For example, cannula (170) may be optically tracked, such as via a fiducial marker (not shown) secured to cannula (170).

### V. EXAMPLE OF A SURGICAL SCOPE WITH RIGID PROXIMAL SHAFT PORTION AND DEFLECTABLE DISTAL SHAFT PORTION CONSTRAINED BY FLEXIBLE OUTER SHEATH

In some instances, it may be desirable to provide a surgical scope that includes a scope shaft having a rigid proximal shaft portion that facilitates insertion and retraction of the scope shaft relative to body cavity (C), while also having a deflectable distal shaft portion that facilitates articulation of the scope shaft to provide visualization of the target surgical site and/or surrounding anatomical structures, and for such a deflectable distal shaft portion to be constrained by a flexible outer sheath. FIGS. 16A-16B show an example of an alternative surgical scope (350) that exhibits such functionality. Surgical scope (350) is similar to surgical scope (150) described above, except as otherwise described below. In this regard, surgical scope (350) includes a scope base (352) having a proximal extension (353), an elongate flexible outer sheath (354) that extends distally from scope base (352), an insertion channel (359) extending through each of scope base (352) and outer sheath (354), and a scope shaft (360) slidably disposed within insertion channel (359). Scope base (352) is configured to attach to drive mechanism (148) of robotic arm head (144) such that drive inputs of scope base (352) operatively couple with respective drive outputs of drive mechanism (148). Drive mechanism (148) is operable to drive insertion (i.e., longitudinal advancement and retraction) and articulation of scope shaft (360) relative to outer sheath (354).

In the example shown, scope shaft (360) includes a rigid proximal shaft portion (361) and a deflectable distal shaft portion (362). Deflectable distal shaft portion (362) may be similar to deflectable distal shaft portion (162) described above. For example, deflectable distal shaft portion (362) includes an articulation section (364) and a distal tip (366) that includes an optical module having a distally facing lens (not shown) configured to provide visualization of a target surgical site with body cavity (C), such that scope shaft (360) may be articulated at its distal articulation section (364) to suitably orient distal tip (366) within body cavity (C) to provide visualization of the target surgical site and/or surrounding anatomical structures.

Surgical scope (350) of the present example further includes a cannula (370) affixed to a distal end of outer sheath (354). Cannula (370) includes a proximal structure in the form of a puck-shaped hub (372) having a closed proximal end, and a distal structure in the form of a tube (374) that extends distally from a distal end of hub (372). The distal end of hub (372) tapers radially inwardly to the proximal end of tube (374), where tube (374) has a smaller maximum outer diameter than hub (372). The interiors of hub (372) and tube (374) cooperate to define a working channel (376) that may be similar to working channel (176) and is sized and configured to slidably receive and guide deflectable distal shaft portion (362) of scope shaft (360) longitudinally therethrough. Outer sheath (354) is affixed to a sidewall of hub (372) to place insertion channel (359) in communication with working channel (376) such that insertion channel (359) is configured to direct scope shaft (360) into working channel (376) along an introductory axis that is angled (e.g., perpendicular) relative to the central primary axis of cannula (370). Hub (372) may include one or more internal guide features configured to guide scope shaft (360) along the transition from outer sheath (354) to cannula tube (374). Such a configuration may provide the interface between outer sheath (354) and hub (372) with a minimal vertical footprint in the workspace above the patient (P). In other words, such a configuration may enable scope base (352) to be positioned by robotic arm head (144) such that hub (372) and at least a distal portion of outer sheath (354) lie within a plane positioned at a minimal height above body wall (W), and such that scope base (352) and robotic arm head (144) may be positioned at or beneath such plane.

Cannula (370) further includes an articulation joint (382) at the distal end of tube (374) that includes a rigid proximal link (384) affixed to the distal end of tube (374), and a rigid distal link (386) pivotably coupled with proximal link (384) about a pivot axis that extends transversely to a primary central axis of cannula (370). Accordingly, articulation joint (382) of the present version is configured to articulate in a single plane, though may be modified to articulate in multiple planes. Additionally, articulation of articulation joint (382) may be active or passive. In cases of active articulation, articulation joint (382) may be driven by drive mechanism (148) of robotic arm head (144), or by another drive mechanism positioned remotely from robotic arm head (144), such as a drive mechanism that is housed within hub (372) and includes one or more motors, for example. Articulation drive may be communicated to articulation joint (382) by one or more tendons (e.g., pull-wires, drive bands, etc.) and/or by any other suitable kind of actuation features.

Insertion of surgical scope (350) is grounded at scope base (352) such that distal tip (366) is configured to selectively move from a retracted position (FIG. 16A) to an extended position (FIG. 16B), vice versa, and any desired longitudinal position therebetween. In this regard, rigid proximal shaft portion (361) is configured to be selectively driven longitudinally through a straight portion of insertion channel (359) proximal of outer sheath (354). Any suitable features may be used to facilitate the driving of rigid proximal shaft portion (361) by drive mechanism (148), including any one or more of pulleys, cables, carriers, such as a kinetic articulating rotating tool (KART), and/or other structures configured to communicate movement to rigid proximal shaft portion (361). As shown, rigid proximal shaft portion (361) is directly connected to deflectable distal shaft portion (362). In this manner, rigid proximal shaft portion (361) is configured to push deflectable distal shaft portion (362) distally when rigid proximal shaft portion (361) is driven distally by drive mechanism (148), and is configured to pull deflectable distal shaft portion (362) proximally when rigid proximal shaft portion (361) is driven proximally by drive mechanism (148). For example, rigid proximal shaft portion (361) may be configured to push deflectable distal shaft portion (362) distally through any bent and/or curved portions of insertion channel (359) within outer sheath (354) and through working channel (376) to thereby extend deflectable distal shaft portion (362) out of working channel (376), and may be configured to pull deflectable distal shaft portion (362) proximally back through working channel (376) and any bent and/or curved portions of insertion channel (359) within outer sheath (354) to thereby retract deflectable distal shaft portion (362) into insertion channel (359).

As mentioned above, outer sheath (354) of the present example is flexible. Thus, outer sheath (354) may define one or more curved and/or bent shapes along the length of outer sheath (354). In some cases, outer sheath (354) may be curved and/or bent to generally conform to a portion of the patient's body. Such a configuration may provide outer sheath (354) with a minimal vertical footprint in the workspace above the patient (P). In other words, such a configuration may enable scope base (352) to be positioned by robotic arm head (144) such that hub (372) and at least a distal portion of outer sheath (354) lie within a plane positioned at a minimal height above body wall (W), and such that the proximal portion of outer sheath (354) may be positioned at or beneath such plane. In addition, or alternatively, the flexibility of outer sheath (354) may permit outer sheath (354) to be moved while cannula (370) remains stationary (e.g., relative to body wall (W)). Outer sheath (354) may have a limited bend-radius flexibility. For example, outer sheath (354) may be bendable between a first configuration in which outer sheath (354) defines a predetermined maximum radius of curvature and a second configuration in which outer sheath (354) defines a predetermined minimum radius of curvature. In some versions, outer sheath (354) is malleable. In still other versions, outer sheath (354) may include a rigidizing feature such that outer sheath (354) is configured to selectively transition between a flexible state and a rigid state. Such a rigidizing feature may include one or more inflatable balloons configured to transition from a deflated state in which outer sheath (354) is flexible to an inflated state in which the one or more balloons imparts rigidity to outer sheath (354), in response to the one or more balloons being filled with an inflation fluid, which may be a liquid or a gas (e.g., air). In addition, or alternatively, such a rigidizing feature may include a plurality of tubular links pivotably coupled with each other to define outer sheath (354) and operatively coupled to corresponding pull cables that are configured to selectively lock the tubular links relative to each other. Surgical scope (350) may be stabilized (or “grounded”) relative to a patient in any suitable manner, such as by use of a separate stabilizing arm or by releasable attachment (e.g., adhesive attachment) to one or more body portions of the patient as described in greater detail below. Alternatively, table (16, 34) or some other structure may be used to mechanically ground surgical scope (350).

It will be appreciated that deflectable distal shaft portion (362) may be constrained by insertion channel (359) when retracted therein to extend generally coaxially with insertion channel (359), and may be permitted to deflect (e.g., articulate) when extended therefrom. More particularly, deflectable distal shaft portion (362) may be constrained by insertion channel (359) to assume a shape corresponding to that of outer sheath (354) when disposed entirely within the insertion channel (259), and to assume a shape corresponding to that of hub (372) and/or tube (374) of cannula (370) when disposed at least partially within working channel (376). Deflectable distal shaft portion (362) may likewise conform to the curvature defined by articulation joint (382) of cannula (370) as scope shaft (360) is guided into body cavity (C). As a portion of scope shaft (360) extends distally beyond the articulated articulation joint (382), that portion of scope shaft (360) resiliently returns to its straight configuration. This distal region of scope shaft (360) may remain straight as scope shaft (360) continues to advance distally relative to articulation joint (382), with the bend in scope shaft (360) being maintained at articulation joint (382).

While cannula (370) of the present example is integrated into the structure of surgical scope (350), cannula (370) may alternatively be configured to releasably couple with surgical scope (350), such as in a manner similar to that described above.

### VI. EXAMPLES OF SURGICAL SCOPE GROUNDING FEATURES

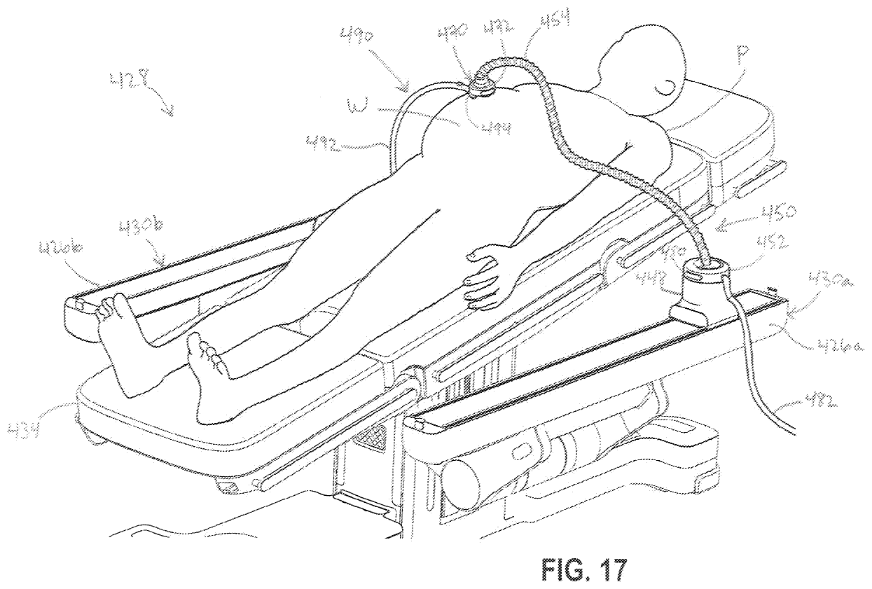

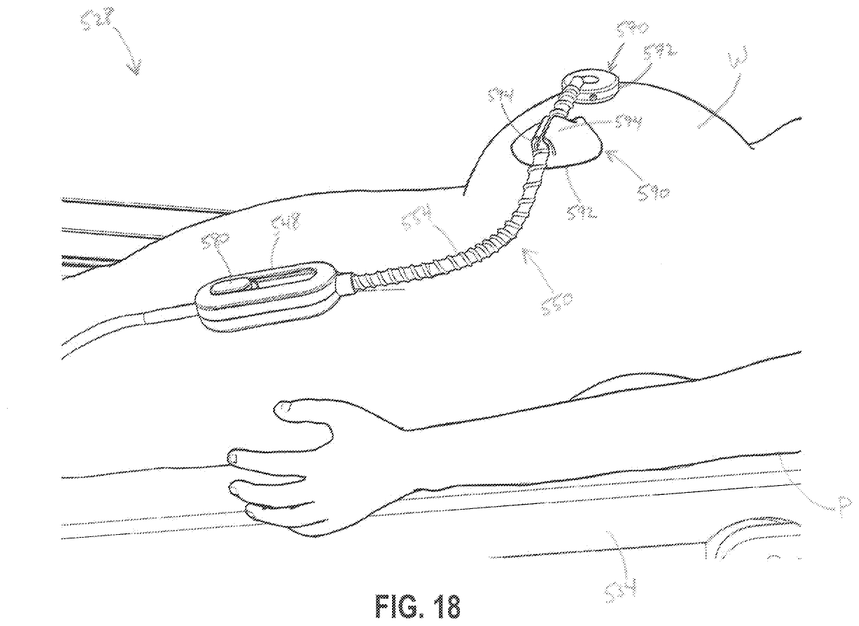

As described above, it may be desirable to stabilize a surgical scope of a robotic system relative to a patient using one or more mechanical grounding features that are independent from the robotic arm (140). FIGS. 17-18 described below show examples of mechanical grounding features in connection with surgical scopes (450, 550), though these grounding features may also be used in connection with any of the other examples of surgical scopes disclosed herein.

A. Example of a Surgical Scope with a Bar-Mounted Stabilizer

FIG. 17 shows another example of a robotic system (428) that includes first and second adjustable arm supports (430a, 430b) mounted on opposite sides of a table (434). System (428), arm supports (430a, 430b), and table (434) may be similar to system (28), arm supports (30), and table (34) described above, respectively, except as otherwise described below. In this regard, first and second arm supports (430a, 430b) include first and second bars (426a, 426b), respectively. System (428) of the present example also includes a surgical scope (450), which is similar to surgical scope (350) described above, except as otherwise described below. In this regard, surgical scope (450) includes a scope base (452) having a proximal extension (not shown), an elongate flexible outer sheath (454) that extends distally from scope base (452), an insertion channel (not shown) extending through each of scope base (452) and outer sheath (454), and a scope shaft (not shown) slidably disposed within the insertion channel. The scope shaft may include a rigid proximal shaft portion and a deflectable distal shaft portion similar to rigid proximal shaft portion (361) and deflectable distal shaft portion (362) described above, respectively. Scope base (452) is configured to attach to a drive mechanism (448), such as via any suitable latching mechanism.

System (428) of the present example further includes a cannula (470) coupled to a distal end of outer sheath (454). Cannula (470) includes a puck-shaped hub (472), and may further include a tube that extends distally from a distal end of hub (472) similar to tube (374) described above, and an articulation joint at a distal end of the tube similar to articulation joint (382) described above. In some versions, cannula (470) may be integrated into surgical scope (450), such as by being permanently coupled to the distal end of outer sheath (454). In some other versions, surgical scope (450) may include a cannula docking plate configured to releasably couple with cannula (470).

Drive mechanism (448) is mounted to first bar (426a) of first arm support (430a), and may be similar to drive mechanism (148) in that drive mechanism (448) is configured to drive articulation of the articulation joint of cannula (470); rotation of the cannula tube relative to hub (472) of cannula (470); advancement and retraction of the scope shaft relative to cannula (470) and outer sheath (454), and/or articulation of a deflectable distal shaft portion of the scope shaft of surgical scope (450). Scope base (452) of the present example includes a user input feature in the form of a toggle switch (480) which may be operatively coupled to drive mechanism (448) to regulate the performance of drive mechanism (448). For example, toggle switch (480) may be configured to facilitate selective longitudinal advancement and retraction of the scope shaft relative to outer sheath (454) via depression of respective sides of toggle switch (480), for example. In addition, or alternatively, actuation of drive mechanism (448) may be performed via control signals received from a master controller (not shown) similar to master controller (132). For example, such a master controller may be operatively coupled to drive mechanism (448) via a cable (482) extending from scope base (452) to the master controller.

In the example shown, system (428) further includes a mechanical grounding member in the form of a stabilizing arm (490) that is mounted to second bar (426b) of second arm support (430b). Stabilizing arm (490) of the present example includes a bent or curved rod (492) having a generally vertical proximal portion that extends upwardly from second bar (426b) and a generally horizontal distal portion that extends laterally at least partially over the body wall (W) of the patient (P). As shown, stabilizing arm (490) further includes a retention mechanism in the form of a generally C-shaped grip (494) extending distally from the distal portion of rod (492) and configured to securely retain an extracorporeal portion of cannula (470) (e.g., hub (472)) during use when the tube of cannula (470) is deployed in the body wall (W). In this regard, rod (492) may have sufficient rigidity to stabilize cannula (470) and thus surgical scope (450) relative to the patient (P).

In some versions, rod (492) may include a rigidizing feature such that rod (492) is configured to selectively transition between a flexible state and a rigid state. Such a rigidizing feature may include one or more inflatable balloons configured to transition from a deflated state in which rod (492) is flexible to an inflated state in which the one or more balloons imparts rigidity to rod (492), in response to the one or more balloons being filled with an inflation fluid, which may be a liquid or a gas (e.g., air). In still other versions, rod (492) is malleable. While grip (494) is shown, any other suitable type of retention mechanism may be used to securely retain a portion of cannula (470), such as a clip, a latch, or one or more clamp arms. In some versions, cannula (470) may be integrated into stabilizing arm (490), such as by being permanently coupled to the distal end of rod (492).

In some instances, stabilizing arm (490) may be configured to securely hold cannula (470) in place relative to the patient (P) irrespective of whether surgical scope (450) is coupled to cannula (470). For example, surgical scope (450) may be released from cannula (470) while cannula (470) remains securely held in place relative to the patient (P) via stabilizing arm (490). Such a configuration may permit a distal tip of the scope shaft to be retracted proximally out of cannula (470) to facilitate cleaning of the distal tip of the scope shaft (e.g., an optical module of the distal tip) while reliably maintaining cannula (470) at the desired location relative to the body wall (W), such that surgical scope (450) may be subsequently recoupled to cannula (470) and the distal tip may be returned to the same position that the distal tip was located at prior to being retracted for cleaning.

Though not shown, robotic system (428) may further include one or more robotic arms (20, 32, 140) each supporting and controlling a respective surgical instrument having an end effector of which surgical scope (450) may provide visualization within a body cavity (C) of the patient (P). Such robotic arm(s) (20, 32, 140) may be supported the bar(s) (426a, 426b) of either arm support (430a, 430b), and the respective surgical instrument(s) may have a relatively large vertical footprint in the workspace above the patient (P). As noted above, drive mechanism (448) may be mounted directly to one of the bars (426a, 426b) of arm supports (430a, 430b) (e.g., rather than being mounted to the one or more robotic arms (20, 32, 140)). Such a configuration may provide the interface between drive mechanism (448) and scope base (452), as well as the rest of surgical scope (450), with a minimal vertical footprint in the workspace above the patient (P). In other words, such a configuration may enable scope base (452) to be positioned along first bar (426a) such that hub (472) and at least a distal portion of outer sheath (454) lie within a plane positioned at a minimal height above body wall (W), and such that scope base (452) and drive mechanism (448) may be positioned at or beneath such plane. In addition, or alternatively, such a configuration may enable any suitable number and/or type of motors for driving insertion and articulation of the scope shaft relative to outer sheath (454) to be incorporated into drive mechanism (448).

B. Example of a Surgical Scope with a Patient-Mounted Stabilizer

FIG. 18 shows another example of a robotic system (528) that includes a table (534). System (528) and table (534) may be similar to system (428) and table (434) described above, respectively, except as otherwise described below. System (528) of the present example also includes a surgical scope (550), which is similar to surgical scope (450) described above, except as otherwise described below. In this regard, surgical scope (550) includes a scope base (not shown), an elongate flexible outer sheath (554) that extends distally from the scope base, an insertion channel (not shown) extending through outer sheath (554), a scope shaft (not shown) slidably disposed within the insertion channel, an integrated cannula (570) affixed to a distal end of outer sheath (554), and a motorized drive mechanism (548) located between the scope base and cannula (570). The scope shaft may include a rigid proximal shaft portion and a deflectable distal shaft portion similar to rigid proximal shaft portion (361) and deflectable distal shaft portion (362) described above, respectively. Cannula (570) includes a puck-shaped hub (572), and may further include a tube that extends distally from a distal end of hub (572) similar to tube (374) described above, and an articulation joint at a distal end of the tube similar to articulation joint (582) described above. In some other versions, surgical scope (550) may include a cannula docking plate configured to releasably couple with cannula (570).

Drive mechanism (548) rests freely on a portion of the patient (P) and may be similar to drive mechanism (148) in that drive mechanism (548) is configured to drive articulation of the articulation joint of cannula (570); rotation of the cannula tube relative to hub (572) of cannula (570); advancement and retraction of the scope shaft relative to cannula (570) and outer sheath (554), and/or articulation of a deflectable distal shaft portion of the scope shaft of surgical scope (550). As shown, drive mechanism (548) is distal to the scope base and proximal to cannula (570), and includes a user input feature in the form of a slidable switch (580) which may be operatively coupled to drive mechanism (548) to regulate the performance of drive mechanism (548). For example, switch (580) may be configured to facilitate selective longitudinal advancement and retraction of the scope shaft relative to outer sheath (554) via sliding of switch (580) in respective directions, for example. In addition, or alternatively, actuation of drive mechanism (548) may be performed via control signals received from a master controller (not shown) similar to master controller (132).

In the example shown, system (528) further includes a mechanical grounding member in the form of a stabilizing patch (590) that is mounted to the body wall (W) of the patient (P). Stabilizing patch (590) of the present example includes a base (592) having a bottom surface that is configured to be releasably adhered to the body wall (W). For example, a suitable adhesive may be applied to the bottom surface of base (592) to facilitate such releasable adhesion to the body wall (W). As shown, stabilizing patch (590) further includes a retention mechanism in the form of an opposed pair of clamp arms (594) extending upwardly from an upper surface of base (592) and configured to securely retain a distal portion of outer sheath (554) during use. In this regard, base (592) may have sufficient rigidity to stabilize outer sheath (554) and thus surgical scope (550) relative to the patient (P). While clamp arms (594) are shown, any other suitable type of retention mechanism may be used to securely retain a portion of outer sheath (554), such as a clip, a latch, or a grip. In some versions, stabilizing patch (590) may be integrated into surgical scope (550), such as by being permanently coupled to the distal portion of outer sheath (554).

In some instances, stabilizing patch (590) may be configured to securely hold a portion of outer sheath (454) in place relative to the patient (P) irrespective of whether surgical scope (550) is coupled to cannula (570). For example, surgical scope (550) may be released from cannula (570) while a portion of outer sheath (454) remains securely held in place relative to the patient (P) via stabilizing patch (590). Such a configuration may permit a distal tip of the scope shaft to be retracted proximally out of cannula (570) to facilitate cleaning of the distal tip of the scope shaft (e.g., an optical module of the distal tip) while reliably maintaining a portion of outer sheath (554) at the desired location relative to the body wall (W), such that surgical scope (550) may be subsequently recoupled to cannula (570) and the distal tip may be returned to the same position that the distal tip was located at prior to being retracted for cleaning.

Though not shown, robotic system (528) may further include one or more robotic arms (20, 32, 140) each supporting and controlling a respective surgical instrument having an end effector of which surgical scope (550) may provide visualization within a body cavity (C) of the patient (P). Such robotic arm(s) (20, 32, 140) may be supported by the bar(s) of one or more arm supports (not shown), and the respective surgical instrument(s) may have a relatively large vertical footprint in the workspace above the patient (P). As noted above, drive mechanism (548) may rest freely on the patient (P) (e.g., rather than being mounted to the one or more robotic arms (20, 32, 140) or to an arm supports). Such a configuration may provide drive mechanism (548) and the rest of surgical scope (550), with a minimal vertical footprint in the workspace above the patient (P). In other words, such a configuration may enable outer sheath (554) to fit closely against the patient (P) such that hub (572) and at least a distal portion of outer sheath (554) lie within a plane positioned at a minimal height above body wall (W), and such that the scope base and drive mechanism (548) may be positioned at or beneath such plane. In addition, or alternatively, such a configuration may allow each of the robotic arm(s) (20, 32, 140) and/or arm supports to be used for purposes other than supporting surgical scope (550). In some versions, drive mechanism (548) may be mounted to the patient (P), such as in a manner similar to that described above with respect to stabilizing patch (590).

### VII. EXAMPLE OF A SURGICAL SCOPE WITH A DUAL-MOUNTING SCOPE BASE

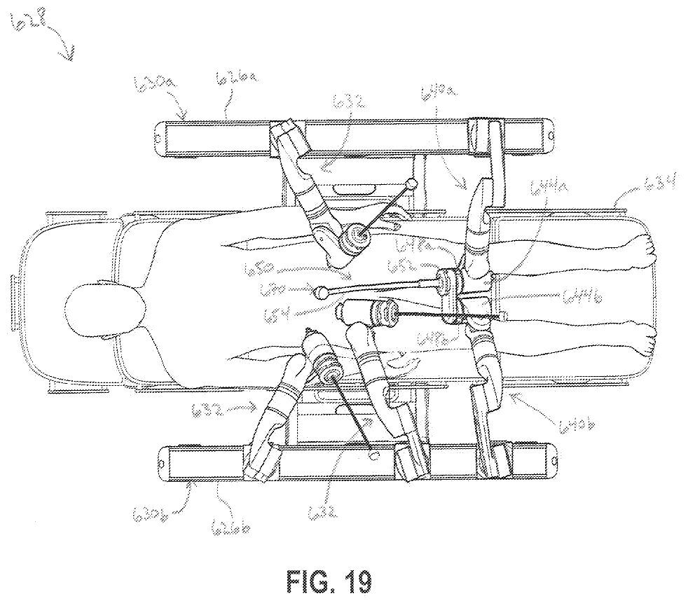

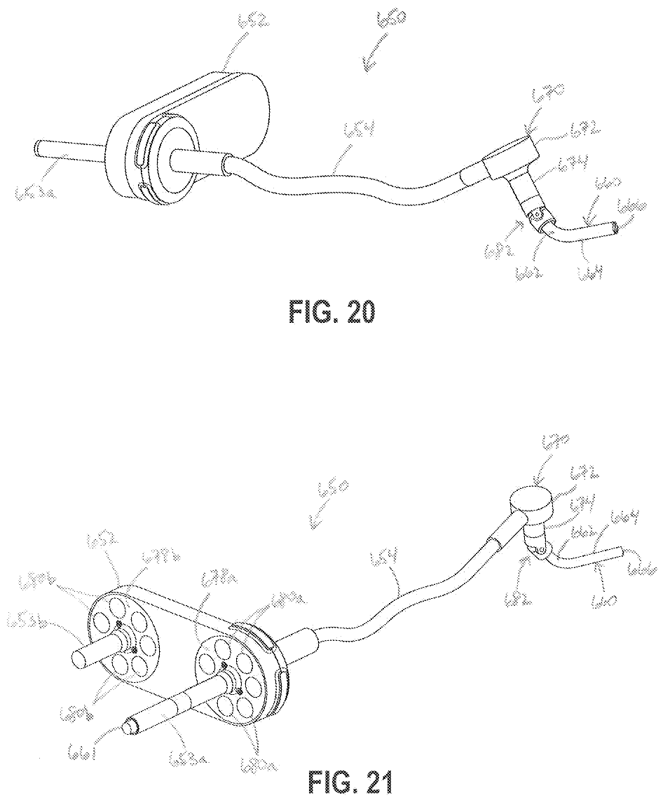

As described above, it may be desirable to provide at least one drive mechanism that is configured to drive articulation of an articulation joint of a cannula; rotation of a cannula tube of the cannula relative to a hub of the cannula; advancement and retraction of a scope shaft of a surgical scope relative to the cannula and an outer sheath of the surgical scope, and/or articulation of a deflectable distal shaft portion of the scope shaft of the surgical scope. In some instances, it may be desirable to provide multiple drive mechanisms, such as two drive mechanisms, to facilitate such types of motion. For example, it may be desirable to provide a first drive mechanism that is configured to drive articulation of the deflectable distal shaft portion of the scope shaft of the surgical scope, and a second drive mechanism that is configured to drive articulation of the articulation joint of the cannula. FIGS. 19-21 described below show examples of dual driving features in connection with surgical scope (650), though these driving features may also be used in connection with any of the other examples of surgical scopes disclosed herein.

FIGS. 19-21 shows another example of a robotic system (628) that includes first and second adjustable arm supports (630a, 630b) mounted on opposite sides of a table (634) and including first and second bars (626a, 626b), respectively, and a plurality of surgical instrument-supporting robotic arms (632) supported by respective bars (626a, 626b) of arm supports (630a, 630b), each supporting and controlling a respective surgical instrument having an end effector. System (628), arm supports (630a, 630b), table (634), and robotic arms (632) may be similar to system (28), arm supports (30), table (34), and robotic arms (32) described above, respectively, except as otherwise described below. System (628) of the present example also includes first and second surgical scope-supporting robotic arms (640a, 640b) supported by first and second bars (626a, 626b) of arm supports (630a, 630b), respectively, and collectively supporting a surgical scope (650).

Robotic arms (640a, 640b) and surgical scope (650) are similar to robotic arms (140) and surgical scope (350) described above, respectively, except as otherwise described below. In this regard, robotic arms (640a, 640b) include respective heads (644a, 644b), where heads (644a, 644b) include respective motorized drive mechanisms (648a, 648b) that may be similar to motorized drive mechanism (148) described above; and surgical scope (650) includes a scope base (652) having first and second proximal extensions (653a, 653b), an elongate flexible outer sheath (654) that extends distally from scope base (652), an insertion channel (not shown) extending through each of scope base (652) and outer sheath (654), a scope shaft (660) slidably disposed within the insertion channel, and an integrated cannula (670) affixed to a distal end of outer sheath (654). Scope shaft (660) includes a rigid proximal shaft portion (661) (FIG. 21) and a deflectable distal shaft portion (662) similar to rigid proximal shaft portion (361) and deflectable distal shaft portion (362) described above, respectively. For example, deflectable distal shaft portion (662) includes an articulation section (664) and a distal tip (666) that includes an optical module having a distally facing lens configured to provide visualization of a target surgical site with body cavity (C). Scope base (652) is configured to attach to first and second drive mechanisms (648a, 648b), such as via any suitable latching mechanisms. Cannula (670) includes a puck-shaped hub (672), a tube (674) that extends distally from a distal end of hub (672), and an articulation joint (682) at a distal end of tube (674). In some other versions, surgical scope (650) may include a cannula docking plate configured to releasably couple with cannula (670).

Scope base (652) of the present example includes first and second attachment surfaces (678a, 678b) and first and second pluralities of drive inputs (680a, 680b) (such as receptacles, pulleys, and spools) configured to receive and couple with respective drive outputs (e.g., rotary drive outputs) of first and second drive mechanisms (648a, 648b), respectively. In this regard, first drive mechanism (648a) may be configured to drive articulation of deflectable distal shaft portion (662). In some cases, first drive mechanism (648a) may also be configured to drive advancement and retraction of scope shaft (660) relative to cannula (670) and outer sheath (654). In addition, or alternatively, second drive mechanism (648b) may be configured to drive articulation of articulation joint (682) of cannula (680). In some cases, second drive mechanism (648b) may also be configured to drive rotation of cannula tube (674) relative to hub (672) of cannula (670). First and second drive mechanisms (648a, 648b) may each include any suitable number and/or type of motors for driving the corresponding types of motion. For example, first drive mechanism (648a) may include four motors. As another example, second drive mechanism (648b) may include between two and four motors.

As noted above, first and second drive mechanisms (648a, 648b) are presented by respective robotic arms (640a, 640b) supported by respective arm supports (630a, 630b). Such a configuration may provide positional control of the insertion (i.e., longitudinal advancement and retraction) of scope shaft (660) relative to outer sheath (654). In addition, or alternatively, such a configuration may allow both drive mechanisms (648a, 648b) to be positioned remotely from the sweeping range of the surgical instruments supported by robotic arms (632). For example, such a configuration may allow each robotic arm (640a, 640b) to be supported by the respective arm support (630a, 630b) at or near an end of the respective bar (628a, 628b), such that drive mechanisms (648a, 648b) may both be positioned on a same side of each of robotic arms (632) (e.g., inferiorly of robotic arms (632) in the frame of reference of the body of the patient (P)). As another example, such a configuration may provide both drive mechanisms (648a, 648b) with a minimal vertical footprint in the workspace above the patient (P).

### VIII. EXAMPLES OF SURGICAL SCOPE SYSTEMS WITH SURGICAL SCOPES AND SEPARATE CANNULA ASSEMBLIES

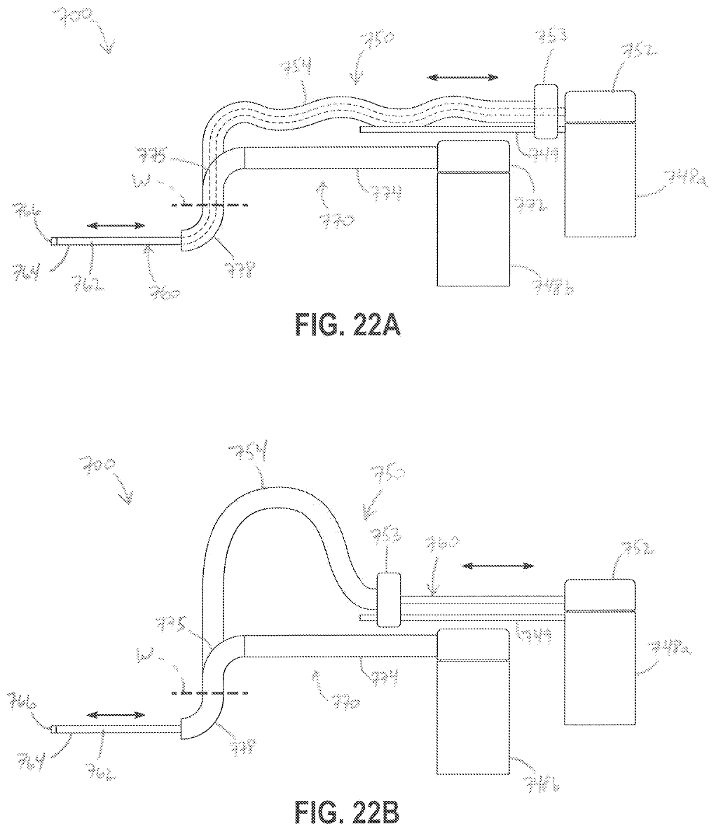

As described above, it may be desirable to provide at least one drive mechanism that is configured to drive articulation of an articulation joint of a cannula; rotation of a cannula tube of the cannula relative to a hub of the cannula; advancement and retraction of a scope shaft of a surgical scope relative to the cannula and an outer sheath of the surgical scope, and/or articulation of a deflectable distal shaft portion of the scope shaft of the surgical scope. In some instances, it may be desirable to incorporate the cannula into a cannula assembly that is separate from the surgical scope, and to equip the cannula assembly with a cannula base that is separate from the scope base and that is configured to attach to a drive mechanism for driving articulation of a distal sheath of the cannula assembly. FIGS. 22A-28B described below show examples of surgical scope systems (700, 800, 900, 1000) that exhibit such functionality.

A. Example of a Surgical Scope System with Side-by-Side Scope Shaft and Cannula Sheath

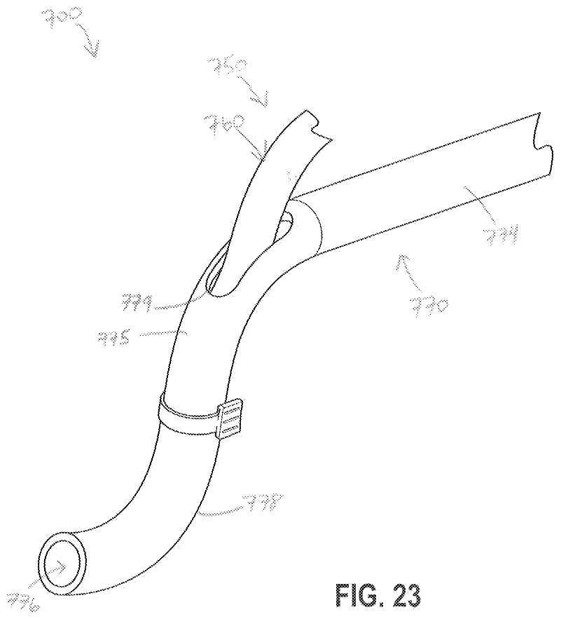

FIGS. 22A-23 show an example of a surgical scope system (700). System (700) of the present example includes a surgical scope (750), which is similar to surgical scope (350) described above, except as otherwise described below. In this regard, surgical scope (750) includes a scope base (752), a sheath base (753), an elongate flexible outer sheath (754) that extends distally from sheath base (753), a first insertion channel (not shown) extending through scope base (752), a second insertion channel (not shown) extending through each of sheath base (753) and outer sheath (754), and a scope shaft (760) slidably disposed within the first and second insertion channels. Scope shaft (760) includes a deflectable distal shaft portion (762). In some cases, scope shaft (760) may include a rigid proximal shaft portion similar to rigid proximal shaft portion (361) described above. Deflectable distal shaft portion (762) may be similar to deflectable distal shaft portion (362) described above. For example, deflectable distal shaft portion (762) includes an articulation section (764) and a distal tip (766) that includes an optical module having a distally facing lens configured to provide visualization of a target surgical site with body cavity (C). Scope base (752) is configured to attach to a first drive mechanism (748a), such as via any suitable latching mechanism. Sheath base (753) is configured to movably attach to first drive mechanism (748a), such as via a rail (749) that is fixed to first drive mechanism (748a) and that sheath base (753) is configured to translate along. In this regard, translation of sheath base (753) along rail (749) may be driven by first drive mechanism (748a), and may be used to adjust a shape of the second insertion channel defined by outer sheath (754), as described in greater detail below.

System (700) of the present example further includes a cannula assembly (770) that includes a cannula base (772), an elongate proximal sheath (774), which may be rigid or deflectable and that extends distally from cannula base (772), and a curved joint (775) at a distal end of proximal sheath (774), which may also be rigid with a predefined curvature or deflectable and configured to assume a curved state as shown. Cannula base (772) is configured to attach to a second drive mechanism (748b), such as via any suitable latching mechanism. Cannula assembly (770) further includes a deflectable distal sheath (778) that extends distally from a distal end of proximal sheath (774) and that is configured to articulate in a manner similar to that described above in connection with distal sheath (106). In this regard, second drive mechanism (748b) may be configured to drive such articulation of distal sheath (778). Cannula assembly (770) of the present example includes a working channel (776) (FIG. 23) extending through each of curved joint (775) and distal sheath (778), such that scope shaft (760) may be slidably disposed within working channel (776).

As best shown in FIG. 23, cannula assembly (770) of the present example further includes a scope port (779) extending through a distal sidewall of curved joint (775) to working channel (776). Scope port (779) of the present example is sized and configured to allow scope shaft (760) to pass therethrough, such that scope shaft (760) may be selectively advanced into and retracted out of a distal region of cannula assembly (770) via scope port (779). Thus, scope port (779) may permit such advancement and retraction of scope shaft (760) relative to cannula assembly (770) while allowing scope shaft (760) to remain external to proximal sheath (774) and/or to be axially offset from (e.g., parallel or obliquely angled relative to) proximal sheath (774). In this regard, a distal end of outer sheath (754) of scope (750) may be securely coupled to scope port (779) so that scope shaft (760) may be guided directly from the second insertion channel into the working channel (776) via scope port (779). With the distal end of outer sheath (754) securely coupled to scope port (779), distal translation of sheath base (753) along rail (749) from a proximal home location may cause outer sheath (754) to assume a generally U-shaped configuration, such that the second insertion channel may likewise be generally U-shaped. It will be appreciated that the angle of approach of scope shaft (760) relative to scope port (779) may vary based on the location of sheath base (753) along rail (749).

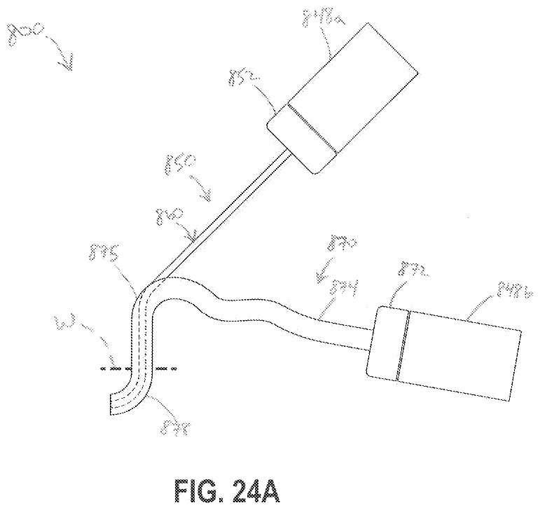

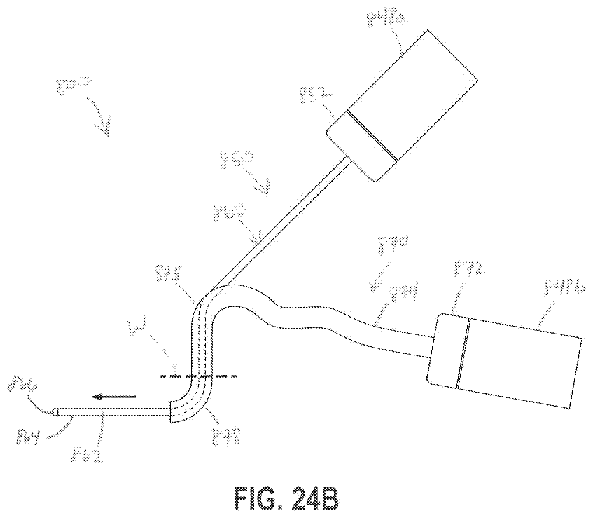

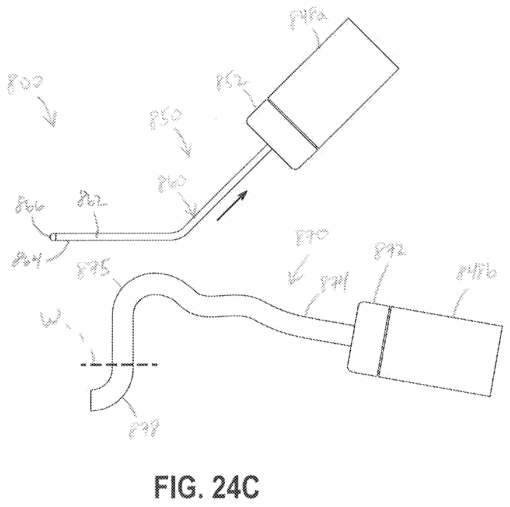

In use, a surgeon may first create an incision in body wall (W), for example at the umbilicus, to provide access to a target surgical site located within body cavity (C). Distal sheath (778) of cannula assembly (770) is inserted distally through the incision in body wall (W) into body cavity (C) while scope shaft (760) remains external to proximal sheath (774), as shown in FIG. 22A. While scope shaft (760) is shown partially disposed within curved joint (775) and distal sheath (778) at this stage in the example shown, scope shaft (760) may alternatively be external to curved joint (775) and/or distal sheath (778) at this stage and may subsequently be advanced into distal sheath (778) via scope port (779). For example, surgical scope (750) may be actuated, for example by first drive mechanism (748a), to translate sheath base (753) distally along rail (749) to provide scope shaft (760) with a desired angle of approach relative to scope port (779), as shown in FIG. 24B. Surgical scope (750) may then be actuated, for example by first drive mechanism (748a), to advance scope shaft (760) distally through curved joint (775) and distal sheath (778) and into body cavity (C), such that distal sheath (778) serves as an introducer cannula. Before, during, or after advancement of scope shaft (760), distal sheath (778) may be articulated by second drive mechanism (748b) to a desired articulated state. Additionally, upon exiting distal sheath (778) and entering body cavity (C), articulation section (764) of scope shaft (760) may be driven by first drive mechanism (748a) to orient distal tip (766) of scope shaft (760) in a desired direction.

B. Examples of Surgical Scope Systems with Efficient Cleaning Accessibility

During a surgical procedure, the distal lens of a surgical scope may accumulate bodily fluids and/or debris that may obstruct or otherwise reduce visualization such that the lens requires cleaning before the procedure can proceed effectively. Accordingly, it may be desirable to provide a surgical scope system that is constructed to enable efficient retraction of the lens for such cleaning, and efficient reinsertion and positioning subsequent to such cleaning. FIGS. 24A-27 described below show examples of surgical scope systems (800, 900) that exhibit such functionality.

i. Example of a Surgical Scope System with Obliquely-Angled Scope Shaft and Cannula Sheath

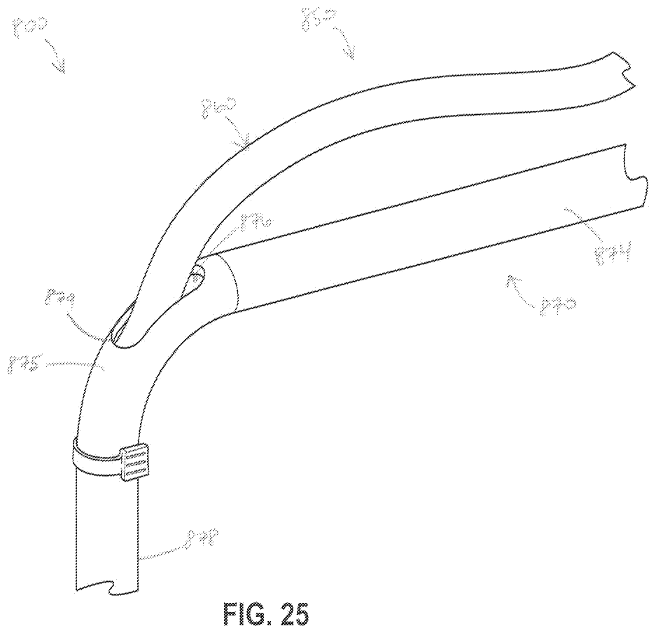

FIGS. 24A-25 show an example of an alternative surgical scope system (800). System (800) of the present example includes a surgical scope (850), which is similar to surgical scope (350) described above, except as otherwise described below. In this regard, surgical scope (850) includes a scope base (852), an insertion channel (not shown) extending through scope base (852), and a scope shaft (860) slidably disposed within the insertion channel. Scope shaft (860) includes a deflectable distal shaft portion (862). In some cases, scope shaft (860) may include a rigid proximal shaft portion similar to rigid proximal shaft portion (361) described above. Deflectable distal shaft portion (862) may be similar to deflectable distal shaft portion (362) described above. For example, deflectable distal shaft portion (862) includes an articulation section (864) and a distal tip (866) that includes an optical module having a distally facing lens configured to provide visualization of a target surgical site with body cavity (C). Scope base (852) is configured to attach to a first drive mechanism (848a), such as via any suitable latching mechanism.

System (800) of the present example further includes a cannula assembly (870) that includes a cannula base (872), an elongate proximal sheath (874), which may be rigid or deflectable and that extends distally from cannula base (872), and a curved joint (875) at a distal end of proximal sheath (874), which may also be rigid with a predefined curvature or deflectable and configured to assume a curved state as shown. Cannula base (872) is configured to attach to a second drive mechanism (848b), such as via any suitable latching mechanism. Cannula assembly (870) further includes a deflectable distal sheath (878) that extends distally from a distal end of proximal sheath (874) and that is configured to articulate in a manner similar to that described above in connection with distal sheath (106). In this regard, second drive mechanism (848b) may be configured to drive such articulation of distal sheath (878). Cannula assembly (870) of the present example includes a working channel (876) (FIG. 25) extending through each of curved joint (875) and distal sheath (878), such that scope shaft (860) may be slidably disposed within working channel (876).

As best shown in FIG. 25, cannula assembly (870) of the present example further includes a scope port (879) extending through a distal sidewall of curved joint (875) to working channel (876). Scope port (879) of the present example is sized and configured to allow scope shaft (860) to pass therethrough, such that scope shaft (860) may be selectively advanced into and retracted out of a distal region of cannula assembly (870) via scope port (879). Thus, scope port (879) may permit such advancement and retraction of scope shaft (860) relative to cannula assembly (870) while allowing scope shaft (860) to remain external to proximal sheath (874) and/or to be axially offset from (e.g., obliquely angled relative to) proximal sheath (874). In this manner, first drive mechanism (848a) may be positioned at a variety of suitable locations relative to second drive mechanism (848b) without interfering with the ability of scope shaft (860) to be advanced through distal sheath (878). For example, first drive mechanism (848a) may be positioned remotely from the sweeping range of any surgical instruments and/or robotic arms.

In use, a surgeon may first create an incision in body wall (W), for example at the umbilicus, to provide access to a target surgical site located within body cavity (C). Distal sheath (878) of cannula assembly (870) is inserted distally through the incision in body wall (W) into body cavity (C) while scope shaft (860) remains external to proximal sheath (874), as shown in FIG. 24A. While scope shaft (860) is shown partially disposed within curved joint (875) and distal sheath (878) at this stage in the example shown, scope shaft (860) may alternatively be external to curved joint (875) and/or distal sheath (878) at this stage and may subsequently be advanced into distal sheath (878) via scope port (879). Surgical scope (850) may then be actuated, for example by first drive mechanism (848a), to advance scope shaft (860) distally through curved joint (875) and distal sheath (878) and into body cavity (C), as shown in FIG. 24B, such that distal sheath (878) serves as an introducer cannula. Before, during, or after advancement of scope shaft (860), distal sheath (878) may be articulated by second drive mechanism (848b) to a desired articulated state. Additionally, upon exiting distal sheath (878) and entering body cavity (C), articulation section (864) of scope shaft (860) may be driven by first drive mechanism (848a) to orient distal tip (866) of scope shaft (860) in a desired direction.

As shown in FIG. 24C, scope port (879) may provide quick and easy access to the lens of distal tip (866) of scope shaft (860) for an intraoperative cleaning or other service, for example. Specifically, scope shaft (860) may be retracted proximally through distal sheath (878) and out of curved joint (875) via scope port (879). Upon retraction of scope shaft (860) from scope port (879), distal tip (866) can be accessed at the distal end of surgical scope (850) for cleaning or other servicing. As shown, articulation section (864) may be articulated to place distal tip (866) at a desired cleaning position. Once complete, scope shaft (860) may be quickly and easily reinserted through curved joint (875) and distal sheath (878) in the same manner described above. Advantageously, this approach avoids having to remove cannula assembly (870) from body wall (W) or having to retract scope shaft (860) fully proximally through cannula assembly (870) (e.g., through a proximal end of proximal sheath (874)) in order to gain access for cleaning or servicing.

Though not shown, cannula assembly (870) may include one or more inner seal members, which may be similar to inner seal member (180), configured to maintain insufflation of body cavity (C) both when surgical scope (850) is partially disposed within and fully removed from cannula assembly (870). For example, such a seal member may be disposed within scope port (879). Accordingly, surgical scope (850) may be withdrawn from cannula assembly (870) during a surgical procedure to access distal tip (866) of surgical scope (850), without disrupting the position of cannula assembly (870) and without compromising insufflation of body cavity (C).

ii. Example of a Surgical Scope System with Coaxial Scope Shaft and Cannula Sheath

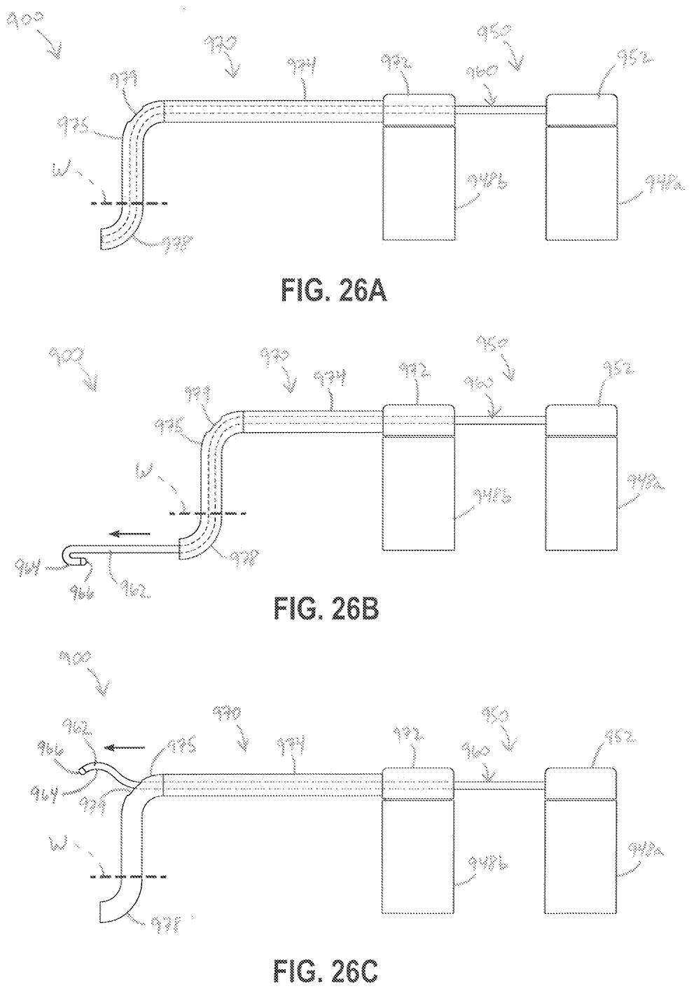

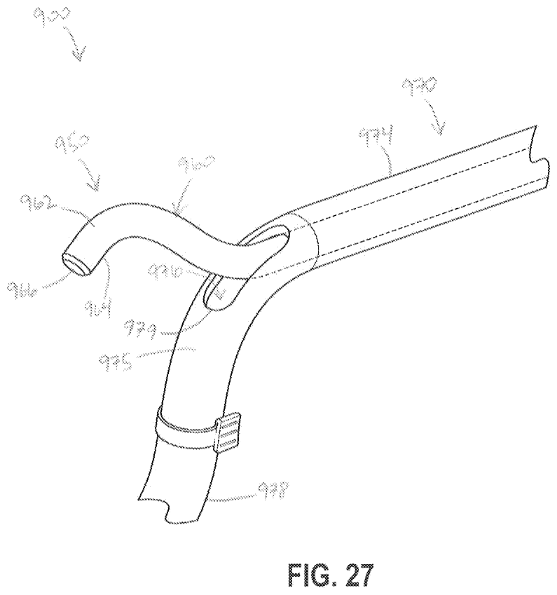

FIGS. 26A-27 show an example of an alternative surgical scope system (900). System (900) of the present example includes a surgical scope (950), which is similar to surgical scope (350) described above, except as otherwise described below. In this regard, surgical scope (950) includes a scope base (952), an insertion channel (not shown) extending through scope base (952), and a scope shaft (960) slidably disposed within the insertion channel. Scope shaft (960) includes a deflectable distal shaft portion (962). In some cases, scope shaft (960) may include a rigid proximal shaft portion similar to rigid proximal shaft portion (361) described above. Deflectable distal shaft portion (962) may be similar to deflectable distal shaft portion (362) described above. For example, deflectable distal shaft portion (962) includes an articulation section (964) and a distal tip (966) that includes an optical module having a distally facing lens configured to provide visualization of a target surgical site with body cavity (C). Scope base (952) is configured to attach to a first drive mechanism (948a), such as via any suitable latching mechanism.

System (900) of the present example further includes a cannula assembly (970) that includes a cannula base (972), an elongate proximal sheath (974), which may be rigid or deflectable and that extends distally from cannula base (972), and a curved joint (975) at a distal end of proximal sheath (974), which may also be rigid with a predefined curvature or deflectable and configured to assume a curved state as shown. Cannula base (972) is configured to attach to a second drive mechanism (948b), such as via any suitable latching mechanism. Cannula assembly (970) further includes a deflectable distal sheath (978) that extends distally from a distal end of proximal sheath (974) and that is configured to articulate in a manner similar to that described above in connection with distal sheath (106). In this regard, second drive mechanism (948b) may be configured to drive such articulation of distal sheath (978). Cannula assembly (970) of the present example includes a working channel (976) (FIG. 27) extending through each of cannula base (972), proximal sheath (974), curved joint (975), and distal sheath (978), such that scope shaft (960) may be slidably disposed within working channel (976).

As best shown in FIG. 27, cannula assembly (970) of the present example further includes a scope port (979) extending through a distal sidewall of curved joint (975) to working channel (976). Scope port (979) of the present example is sized and configured to allow scope shaft (960) to pass therethrough, such that scope shaft (960) may be selectively advanced out of and retracted into a distal region of cannula assembly (970) via scope port (979). Thus, scope port (979) may permit such advancement and retraction of scope shaft (960) relative to cannula assembly (970) while allowing scope shaft (960) to remain at least partially disposed within proximal sheath (974) and/or to be axially aligned with proximal sheath (974). As shown, scope shaft (960) may be disposed coaxially within working channel (976) such that first drive mechanism (948a) may be positioned directly behind (e.g., proximal of) second drive mechanism (948b) for advancement scope shaft (960) distally into working channel (976) through cannula base (972).

In use, a surgeon may first create an incision in body wall (W), for example at the umbilicus, to provide access to a target surgical site located within body cavity (C). Distal sheath (978) of cannula assembly (970) is inserted distally through the incision in body wall (W) into body cavity (C), as shown in FIG. 26A. While scope shaft (960) is shown partially disposed within proximal sheath (974), curved joint (975) and distal sheath (978) at this stage in the example shown, scope shaft (960) may alternatively be external to proximal sheath (974), curved joint (975) and/or distal sheath (978) at this stage and may subsequently be advanced into distal sheath (978) via proximal sheath (974) and curved joint (975). Surgical scope (950) may then be actuated, for example by first drive mechanism (948a), to advance scope shaft (960) distally through proximal sheath (974), curved joint (975), and distal sheath (978) and into body cavity (C), as shown in FIG. 26B, such that distal sheath (978) serves as an introducer cannula. Before, during, or after advancement of scope shaft (960), distal sheath (978) may be articulated by second drive mechanism (948b) to a desired articulated state. Additionally, upon exiting distal sheath (978) and entering body cavity (C), articulation section (964) of scope shaft (960) may be driven by first drive mechanism (948a) to orient distal tip (966) of scope shaft (960) in a desired direction, as also shown in FIG. 26B.

As shown in FIG. 26C, scope port (979) may provide quick and easy access to the lens of distal tip (966) of scope shaft (960) for an intraoperative cleaning or other service, for example. Specifically, scope shaft (960) may be retracted proximally through distal sheath (978) and subsequently advanced distally out of curved joint (975) via scope port (979). Upon advancement of scope shaft (960) through scope port (979), distal tip (966) can be accessed at the distal end of surgical scope (950) for cleaning or other servicing. As shown, articulation section (964) may be articulated to place distal tip (966) at a desired cleaning position. Once complete, scope shaft (960) may be quickly and easily reinserted through curved joint (975) and distal sheath (978) in the same manner described above. Advantageously, this approach avoids having to remove cannula assembly (970) from body wall (W) or having to retract scope shaft (960) fully proximally through cannula assembly (970) (e.g., through a proximal end of proximal sheath (974)) in order to gain access for cleaning or servicing.

In some versions, a first position that distal tip (966) of scope shaft (960) is located at for visualizing the target tissue site prior to being retracted for cleaning may be stored in a memory of a master controller (not shown) similar to master controller (132). A second position that distal tip (966) is desired to be located at for cleaning may be preset by the operator prior to beginning the surgical procedure and may likewise be stored in a memory of the master controller. A first user input feature, such as a removal button, may be operatively coupled to each of the master controller and first drive mechanism (948a), such that actuation of the first user input feature may cause first drive mechanism (948a) to automatically retract distal tip (966) from the first position into curved joint (975), advance distal tip (966) out through scope port (979), and articulate deflectable distal shaft portion (962) to place distal tip (966) at the second position for cleaning. A second user input feature, such as a reinsertion button, may likewise be operatively coupled to each of the master controller and first drive mechanism (948a), such that actuation of the second user input feature after cleaning may cause first drive mechanism (948a) to automatically retract distal tip (966) from the second position into curved joint (975) through scope port (979), advance distal tip (966) out through distal sheath (978), and articulate deflectable distal shaft portion (962) to place distal tip (966) at the first position to thereby resume visualizing the target tissue site.

Though not shown, cannula assembly (970) may include one or more inner seal members, which may be similar to inner seal member (180), configured to maintain insufflation of body cavity (C) when surgical scope (950) is disposed within curved joint (975) without passing through scope port (979), when surgical scope (950) passes through scope port (979), and when surgical scope (950) is fully removed from cannula assembly (970). For example, such a seal member may be disposed within scope port (979). Accordingly, surgical scope (950) may be advanced through scope port (979) during a surgical procedure to access distal tip (966), without disrupting the position of cannula assembly (970) and without compromising insufflation of body cavity (C).

C. Example of a Surgical Scope System with Scope Shaft Feed Rollers and Drive Mechanism Having Optical Support Components

As described above, it may be desirable to provide at least one drive mechanism that is configured to drive articulation of an articulation joint of a cannula; rotation of a cannula tube of the cannula relative to a hub of the cannula; advancement and retraction of a scope shaft of a surgical scope relative to the cannula and an outer sheath of the surgical scope, and/or articulation of a deflectable distal shaft portion of the scope shaft of the surgical scope. In some instances, it may be desirable to configure the drive mechanism to reliably and consistently drive advancement and retraction of the scope shaft to position the distal tip of the scope shaft at one or more predetermined locations. In addition, or alternatively, it may be desirable to provide a separate drive mechanism that includes various data and/or optical support components that support the functionality of the optical module of the distal tip of the scope shaft. For example, such support components may include an optical return path for receiving optical signals from the optical module (e.g., for processing by the master controller to display the captured images), a light source path for supplying light from a light engine (e.g., including white and/or near infrared light sources) to the optical module, and/or data connections for allowing command signals to be transmitted from the master controller to the optical module.

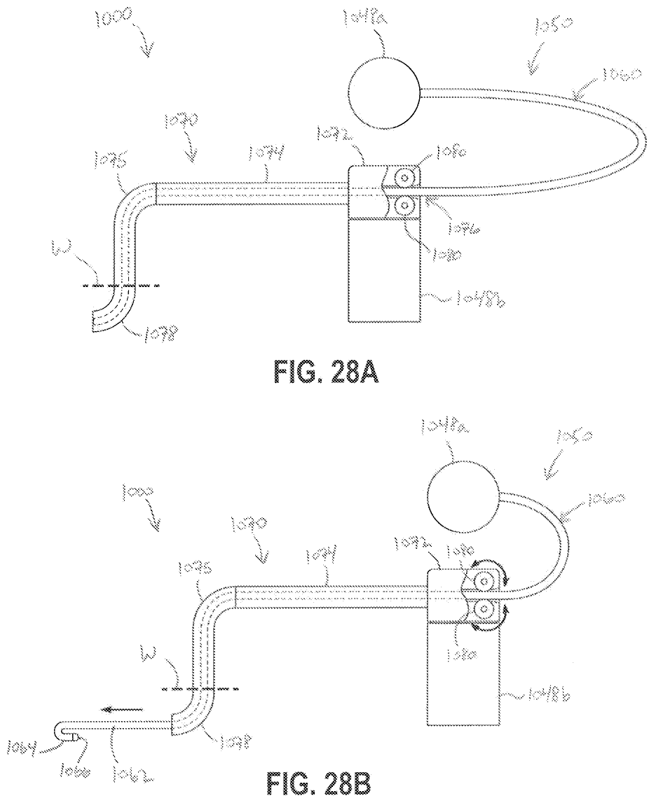

FIGS. 28A-28B show an example of an alternative surgical scope system (1000) that exhibits such functionality. System (1000) of the present example includes a surgical scope (1050), which is similar to surgical scope (350) described above, except as otherwise described below. In this regard, surgical scope (1050) includes a scope base (not shown) and a scope shaft (1060) extending outwardly from the scope base. Scope shaft (1060) includes a deflectable distal shaft portion (1062). Deflectable distal shaft portion (1062) may be similar to deflectable distal shaft portion (362) described above. For example, deflectable distal shaft portion (1062) includes an articulation section (1064) and a distal tip (1066) that includes an optical module having a distally facing lens configured to provide visualization of a target surgical site with body cavity (C). In some cases, scope shaft (1060) may be flexible along an entire length of scope shaft (1060). The scope base is configured to attach to a first drive mechanism (1048a), such as via any suitable latching mechanism. First drive mechanism (1048a) may include any one or more data and/or optical support components that support the functionality of the optical module of distal tip (1066) of scope shaft (1060). For example, first drive mechanism (1048a) may include an optical return path for receiving optical signals from the optical module of distal tip (1066), such as for processing by a master controller similar to master controller (132) to display the images captured by the optical module. In addition, or alternatively, first drive mechanism (1048a) may include a light source path for supplying light from a light engine, which may include any one or more white and/or near infrared light sources, to the optical module of distal tip (1066) for illuminating the target tissue site. In some versions, first drive mechanism (1048a) may include any suitable number of data connections for allowing command signals to be transmitted from the master controller to the optical module of distal tip (1066).

System (1000) of the present example further includes a cannula assembly (1070) that includes a cannula base (1072), an elongate proximal sheath (1074), which may be rigid or deflectable and that extends distally from cannula base (1072), and a curved joint (1075) at a distal end of proximal sheath (1074), which may also be rigid with a predefined curvature or deflectable and configured to assume a curved state as shown. Cannula base (1072) is configured to attach to a second drive mechanism (1048b), such as via any suitable latching mechanism. Cannula assembly (1070) further includes a deflectable distal sheath (1078) that extends distally from a distal end of proximal sheath (1074) and that is configured to articulate in a manner similar to that described above in connection with distal sheath (106). In this regard, second drive mechanism (1048b) may be configured to drive such articulation of distal sheath (1078). Cannula assembly (1070) of the present example includes a working channel (1076) extending through each of cannula base (1072), proximal sheath (1074), curved joint (1075), and distal sheath (1078), such that scope shaft (1060) may be slidably disposed within working channel (1076).

As shown, cannula assembly (1070) of the present example further includes a pair of scope shaft feed rollers (1080) housed within cannula base (1072) and positioned opposite each other relative to working channel (1076) for gripping opposite sides of scope shaft (1060). Feed rollers (1080) may be configured to rotate about respective axes in opposing directions to thereby facilitate advancement and retraction of scope shaft (1060) relative to each of proximal sheath (1074), curved joint (1075), and distal sheath (1078). In this regard, feed rollers (1080) may be operatively coupled with respective outputs of second drive mechanism (1048b), such that second drive mechanism (1048b) may be configured to drive advancement and retraction of scope shaft (1060). In some versions, feed rollers (1080) may be incorporated directly into second drive mechanism (1048b). As shown, scope shaft (1060) may have sufficient length and flexibility to extend outwardly from the scope base in a generally proximal direction and form a generally retrograde (e.g., U-shaped) bend to extend in a generally distal direction for entering working channel (1076) through cannula base (1072). The portion of scope shaft (1060) between the scope base and cannula base (1072) may define a slack or service loop, the length of which may vary during insertion of scope shaft (1060).

In use, a surgeon may first create an incision in body wall (W), for example at the umbilicus, to provide access to a target surgical site located within body cavity (C). Distal sheath (1078) of cannula assembly (1070) is inserted distally through the incision in body wall (W) into body cavity (C), as shown in FIG. 28A. While scope shaft (1060) is shown partially disposed within proximal sheath (1074), curved joint (1075) and distal sheath (1078) at this stage in the example shown, scope shaft (1060) may alternatively be external to proximal sheath (1074), curved joint (1075) and/or distal sheath (1078) at this stage and may subsequently be advanced into distal sheath (1078) via proximal sheath (1074) and curved joint (1075). Surgical scope (1050) may then be actuated, for example by second drive mechanism (1048b) and feed rollers (1080), to advance scope shaft (1060) distally through proximal sheath (1074), curved joint (1075), and distal sheath (1078) and into body cavity (C), as shown in FIG. 28B, such that distal sheath (1078) serves as an introducer cannula. Before, during, or after advancement of scope shaft (1060), distal sheath (1078) may be articulated by second drive mechanism (1048b) to a desired articulated state. Additionally, upon exiting distal sheath (1078) and entering body cavity (C), articulation section (1064) of scope shaft (1060) may be driven by first drive mechanism (1048a) to orient distal tip (1066) of scope shaft (1060) in a desired direction, as also shown in FIG. 28B.

### IX. EXAMPLES OF COMBINATIONS

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

An apparatus, comprising: (a) a scope base configured to be positioned extracorporeally relative to a patient, wherein the scope base is configured to attach to at least one drive mechanism; (b) an outer sheath extending distally from the scope base; (c) an insertion channel extending through each of the scope base and the outer sheath; and (d) a scope shaft actuatable relative to the scope base and slidably disposed within the insertion channel, wherein the scope shaft includes: (i) a rigid proximal shaft portion configured to be driven by the at least one drive mechanism, (ii) a deflectable distal shaft portion, wherein the deflectable distal shaft portion is deflectable relative to the proximal shaft portion, and (iii) a distal end configured to provide visualization of a body cavity.

### Example 2

The apparatus of Example 1, further comprising a cannula, wherein the cannula includes: (i) a proximal structure configured to be positioned extracorporeally relative to a patient, and (ii) a distal structure extending distally from the proximal structure and configured to be passed through a body wall and into a body cavity of the patient, wherein the distal structure cooperates with the primary structure to define a primary axis and a working channel sized and configured to receive and guide the deflectable distal shaft portion distally therethrough along the primary axis.

### Example 3

The apparatus of Example 2, wherein the outer sheath is releasably coupled to the cannula.

### Example 4

The apparatus of Example 2, wherein the outer sheath is integrally formed with the cannula.

### Example 5

The apparatus of any one or more of Examples 2 through 4, wherein the cannula further includes an angled feature at a distal end of the distal structure, wherein the angled feature is configured to direct the deflectable distal shaft portion along a secondary axis that is angled relative to the primary axis.

### Example 6

The apparatus of any one or more of Examples 2 through 4, wherein the cannula further includes an articulation feature at a distal end of the distal structure, wherein the articulation feature is configured to articulate relative to the proximal structure to direct the deflectable distal shaft portion along a secondary axis that is angled relative to the primary axis.

### Example 7

The apparatus of Example 6, wherein the scope base is configured to attach to first and second drive mechanisms, wherein the rigid proximal shaft portion of the scope shaft is configured to be driven by the first drive mechanism, wherein the articulation feature is configured to be driven by the second drive mechanism.

### Example 8

The apparatus of any one or more of Examples 1 through 7, wherein the outer sheath is rigid.

### Example 9

The apparatus of any one or more of Examples 1 through 7, wherein the outer sheath is flexible.

### Example 10

The apparatus of any one or more of Examples 1 through 7, wherein the outer sheath is configured to selectively transition between a flexible state and a rigid state.

### Example 11

A system, comprising: (a) the apparatus of any one or more of Examples 1 through 10; and (b) a mechanical grounding member configured to stabilize a distal portion of the outer sheath relative to a body wall of the patient.

### Example 12

The system of Example 11, wherein the mechanical grounding member includes a stabilizing arm.

### Example 13

The system of Example 12, wherein the stabilizing arm is configured to selectively transition between a flexible state and a rigid state.

### Example 14

The system of Example 11, wherein the mechanical grounding member includes a stabilizing patch configured to mount to the body wall of the patient.

### Example 15

The system of Example 14, wherein the stabilizing patch includes an adhesive applied to a bottom surface of the stabilizing patch for adhering the stabilizing patch to the body wall of the patient.

### Example 16

An apparatus, comprising: (a) a scope base configured to be positioned extracorporeally relative to a patient; (b) an outer sheath extending distally from the scope base; (c) an insertion channel extending through each of the scope base and the outer sheath; (d) a scope shaft actuatable relative to the scope base and slidably disposed within the insertion channel, wherein the scope shaft includes a deflectable shaft portion; and (e) a mechanical grounding member configured to stabilize a distal portion of the outer sheath relative to a body wall of the patient, wherein the mechanical grounding member is operatively coupled to the outer sheath extracorporeally.

### Example 17

The apparatus of Example 16, wherein the mechanical grounding member includes at least one of a stabilizing arm or a stabilizing patch.

### Example 18

The apparatus of any one or more of Examples 16 through 17, wherein the outer sheath is configured to selectively transition between a flexible state and a rigid state.

### Example 19

A system, comprising: (a) a support structure; (b) a motorized drive mechanism coupled to the support structure; (c) a surgical scope, including: (i) a scope base coupled with the motorized drive mechanism, and (ii) a scope shaft that is actuatable relative to the scope base by the motorized drive mechanism, wherein the scope shaft includes: (A) a rigid proximal shaft portion, and (B) a deflectable distal shaft portion; and (d) a cannula, including: (i) a proximal structure configured to be positioned extracorporeally relative to a patient, (ii) a distal structure extending distally from the proximal structure and configured to be passed through a body wall and into a body cavity of the patient, wherein the distal structure defines a primary axis and a working channel sized and configured to receive and guide the deflectable distal shaft portion therethrough along the primary axis, and (iii) a deflection feature configured to direct the deflectable distal shaft portion along a secondary axis that is angled relative to the primary axis.

### Example 20

The system of Example 19, further comprising a mechanical grounding member configured to stabilize a distal portion of the outer sheath relative to the body wall of the patient.

### Example 21

A system, comprising: (a) a surgical scope, including: (i) a scope base configured to be positioned extracorporeally relative to a patient, and (ii) a scope shaft actuatable relative to the scope base, wherein the scope shaft includes a deflectable shaft portion; and (b) a cannula assembly configured to guide the scope shaft relative to the patient, the cannula assembly including: (i) a cannula base configured to be positioned extracorporeally relative to the patient, (ii) a proximal cannula sheath extending distally from the cannula base, (iii) a curved joint at a distal end of the proximal cannula sheath, (iv) a deflectable distal cannula sheath extending distally from the curved joint and configured to be passed through a body wall and into a body cavity of the patient, and (v) a scope port extending through the curved joint, wherein the scope port is sized and configured to allow the scope shaft to pass therethrough.

### Example 22

The system of Example 21, wherein the scope shaft is external to the proximal cannula sheath.

### Example 23

The system of Example 22, wherein the scope shaft is configured to be advanced distally into the curved joint and through the deflectable distal cannula sheath via the scope port.

### Example 24

The system of any one or more of Examples 22 through 23, wherein the scope shaft is configured to be retracted proximally through the deflectable distal cannula sheath and out of the curved joint via the scope port.

### Example 25

The system of any one or more of Examples 22 through 24, wherein the scope shaft is oriented obliquely relative to the proximal cannula sheath.

### Example 26

The system of any one or more of Examples 21 through 25, wherein the surgical scope further includes: (i) a sheath base actuatable relative to the scope base, and (ii) a scope sheath extending distally from the sheath base and having a distal end securely coupled to the scope port, wherein the scope shaft is slidably disposed within the scope sheath.

### Example 27

The system of Example 21, wherein the scope shaft is slidably disposed within the proximal cannula sheath.

### Example 28

The system of Example 27, wherein the scope shaft is configured to be advanced distally through the proximal cannula sheath and out of the curved joint via the scope port.

### Example 29

The system of any one or more of Examples 27 through 28, wherein the scope shaft is configured to be retracted proximally into the curved joint and through the proximal cannula sheath via the scope port.

### Example 30

The system of any one or more of Examples 27 through 29, wherein the scope shaft is coaxial with the proximal cannula sheath.

### Example 31

A system, comprising: (a) first and second support structures; (b) first and second motorized drive mechanisms coupled to the first and second support structures, respectively; (c) a surgical scope, including: (i) a scope base coupled with the first motorized drive mechanism, and (ii) a scope shaft that is actuatable relative to the scope base by the first motorized drive mechanism, wherein the scope shaft includes a deflectable shaft portion; and (d) a cannula assembly configured to guide the scope shaft relative to the patient, the cannula assembly including: (i) a cannula base configured to be positioned extracorporeally relative to the patient, (ii) a cannula sheath extending distally from the cannula base and configured to be passed through a body wall and into a body cavity of the patient, wherein the scope shaft is slidably disposed within the cannula sheath, and (iii) at least one scope shaft feed member housed within the cannula base and operatively coupled with the second drive mechanism for actuating the scope shaft relative to the scope base.

### Example 32

The system of Example 31, wherein the at least one scope shaft feed member includes a pair of scope shaft feed rollers.

### Example 33

The system of any one or more of Examples 31 through 32, wherein the scope shaft includes an optical module, wherein the first drive mechanism includes at least one optical support component in operative communication with the optical module of the distal tip of scope shaft.

### Example 34

The system of Example 33, wherein the at least one optical support component includes an optical return path for receiving optical signals from the optical module.

### Example 35

The system of any one or more of Examples 33 through 34, wherein the at least one optical support component includes a light source path for supplying light from a light engine to the optical module.

### X. MISCELLANEOUS

The teachings herein may be combined with any one or more of the teachings disclosed in U.S. patent application Ser. No. 17/941,063, entitled “Articulating Introducer Cannula for Surgical Scope in Robotic System,” filed Sep. 9, 2022, the disclosure of which is incorporated by reference herein in its entirety; U.S. patent application Ser. No. 17/941,062, entitled “Bent Introducer Cannula for Surgical Scope in Robotic System,” filed Sep. 9, 2022, the disclosure of which is incorporated by reference herein in its entirety; U.S. patent application Ser. No. 17/941,059 entitled “Flexible Articulating Introducer Cannula for Surgical Scope in Robotic System,” filed Sep. 9, 2022, the disclosure of which is incorporated by reference herein in its entirety; U.S. patent application Ser. No. 17/941,057, entitled “Remotely Driven Camera in Robotic System,” filed Sep. 9, 2022, the disclosure of which is incorporated by reference herein in its entirety; and/or U.S. Pat. App. No. [Atty. Ref. AUR6286USNP1], entitled “System and Method to Position Surgical Scope in Robotic System,” filed on even date herewith, the disclosure of which is incorporated by reference herein in its entirety.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

Some versions of the examples described herein may be implemented using a processor, which may be part of a computer system and communicate with a number of peripheral devices via bus subsystem. Versions of the examples described herein that are implemented using a computer system may be implemented using a general-purpose computer that is programmed to perform the methods described herein. Alternatively, versions of the examples described herein that are implemented using a computer system may be implemented using a specific-purpose computer that is constructed with hardware arranged to perform the methods described herein. Versions of the examples described herein may also be implemented using a combination of at least one general-purpose computer and at least one specific-purpose computer.

In versions implemented using a computer system, each processor may include a central processing unit (CPU) of a computer system, a microprocessor, an application-specific integrated circuit (ASIC), other kinds of hardware components, and combinations thereof. A computer system may include more than one type of processor. The peripheral devices of a computer system may include a storage subsystem including, for example, memory devices and a file storage subsystem, user interface input devices, user interface output devices, and a network interface subsystem. The input and output devices may allow user interaction with the computer system. The network interface subsystem may provide an interface to outside networks, including an interface to corresponding interface devices in other computer systems. User interface input devices may include a keyboard; pointing devices such as a mouse, trackball, touchpad, or graphics tablet; a scanner; a touch screen incorporated into the display; audio input devices such as voice recognition systems and microphones; and other types of input devices. In general, use of the term “input device” is intended to include all possible types of devices and ways to input information into computer system.

In versions implemented using a computer system, a storage subsystem may store programming and data constructs that provide the functionality of some or all of the modules and methods described herein. These software modules may be generally executed by the processor of the computer system alone or in combination with other processors. Memory used in the storage subsystem may include a number of memories including a main random-access memory (RAM) for storage of instructions and data during program execution and a read only memory (ROM) in which fixed instructions are stored. A file storage subsystem may provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem in the storage subsystem, or in other machines accessible by the processor.

In versions implemented using a computer system, the computer system itself may be of varying types including a personal computer, a portable computer, a workstation, a computer terminal, a network computer, a television, a mainframe, a server farm, a widely-distributed set of loosely networked computers, or any other data processing system or user device. Due to the ever-changing nature of computers and networks, the example of the computer system described herein is intended only as a specific example for purposes of illustrating the technology disclosed. Many other configurations of a computer system are possible having more or fewer components than the computer system described herein.

As an article of manufacture, rather than a method, a non-transitory computer readable medium (CRM) may be loaded with program instructions executable by a processor. The program instructions when executed, implement one or more of the computer-implemented methods described above. Alternatively, the program instructions may be loaded on a non-transitory CRM and, when combined with appropriate hardware, become a component of one or more of the computer-implemented systems that practice the methods disclosed.

Versions described above may be designed to be disposed of after a single use, or they can be designed to be used multiple times. Versions may, in either or both cases, be reconditioned for reuse after at least one use. Reconditioning may include any combination of the steps of disassembly of the systems, instruments, and/or portions thereof, followed by cleaning or replacement of particular pieces, and subsequent reassembly. In particular, some versions of the systems, instruments, and/or portions thereof may be disassembled, and any number of the particular pieces or parts of the systems, instruments, and/or portions thereof may be selectively replaced or removed in any combination. Upon cleaning and/or replacement of particular parts, some versions of the systems, instruments, and/or portions thereof may be reassembled for subsequent use either at a reconditioning facility, or by an operator immediately prior to a procedure. Those skilled in the art will appreciate that reconditioning of systems, instruments, and/or portions thereof may utilize a variety of techniques for disassembly, cleaning/replacement, and reassembly. Use of such techniques, and the resulting reconditioned systems, instruments, and/or portions thereof, are all within the scope of the present application.

By way of example only, versions described herein may be sterilized before and/or after a procedure. In one sterilization technique, the systems, instruments, and/or portions thereof is placed in a closed and sealed container, such as a plastic or TYVEK bag. The container and system, instrument, and/or portion thereof may then be placed in a field of radiation that can penetrate the container, such as gamma radiation, x-rays, or high-energy electrons. The radiation may kill bacteria on the system, instrument, and/or portion thereof and in the container. The sterilized systems, instruments, and/or portions thereof may then be stored in the sterile container for later use. Systems, instruments, and/or portions thereof may also be sterilized using any other technique known in the art, including but not limited to beta or gamma radiation, ethylene oxide, or steam.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

1. An apparatus, comprising:
(a) a scope base configured to be positioned extracorporeally relative to a patient, wherein the scope base is configured to attach to at least one drive mechanism;
(b) an outer sheath extending distally from the scope base;
(c) an insertion channel extending through each of the scope base and the outer sheath; and
(d) a scope shaft actuatable relative to the scope base and slidably disposed within the insertion channel, wherein the scope shaft includes:
(i) a rigid proximal shaft portion configured to be driven by the at least one drive mechanism,
(ii) a deflectable distal shaft portion, wherein the deflectable distal shaft portion is deflectable relative to the proximal shaft portion, and
(iii) a distal end configured to provide visualization of a body cavity.

2. The apparatus of claim 1, further comprising a cannula, wherein the cannula includes:
(i) a proximal structure configured to be positioned extracorporeally relative to a patient, and
(ii) a distal structure extending distally from the proximal structure and configured to be passed through a body wall and into a body cavity of the patient, wherein the distal structure cooperates with the primary structure to define a primary axis and a working channel sized and configured to receive and guide the deflectable distal shaft portion distally therethrough along the primary axis.

3. The apparatus of claim 2, wherein the outer sheath is releasably coupled to the cannula.

4. The apparatus of claim 2, wherein the outer sheath is integrally formed with the cannula.

5. The apparatus of claim 2, wherein the cannula further includes an angled feature at a distal end of the distal structure, wherein the angled feature is configured to direct the deflectable distal shaft portion along a secondary axis that is angled relative to the primary axis.

6. The apparatus of claim 2, wherein the cannula further includes an articulation feature at a distal end of the distal structure, wherein the articulation feature is configured to articulate relative to the proximal structure to direct the deflectable distal shaft portion along a secondary axis that is angled relative to the primary axis.

7. The apparatus of claim 6, wherein the scope base is configured to attach to first and second drive mechanisms, wherein the rigid proximal shaft portion of the scope shaft is configured to be driven by the first drive mechanism, wherein the articulation feature is configured to be driven by the second drive mechanism.

8. The apparatus of claim 1, wherein the outer sheath is rigid.

9. The apparatus of claim 1, wherein the outer sheath is flexible.

10. The apparatus of claim 1, wherein the outer sheath is configured to selectively transition between a flexible state and a rigid state.

11. A system, comprising:
(a) the apparatus of claim 1; and
(b) a mechanical grounding member configured to stabilize a distal portion of the outer sheath relative to a body wall of the patient.

12. The system of claim 11, wherein the mechanical grounding member includes a stabilizing arm.

13. The system of claim 12, wherein the stabilizing arm is configured to selectively transition between a flexible state and a rigid state.

14. The system of claim 11, wherein the mechanical grounding member includes a stabilizing patch configured to mount to the body wall of the patient.

15. The system of claim 14, wherein the stabilizing patch includes an adhesive applied to a bottom surface of the stabilizing patch for adhering the stabilizing patch to the body wall of the patient.

16. An apparatus, comprising:
(a) a scope base configured to be positioned extracorporeally relative to a patient;
(b) an outer sheath extending distally from the scope base;
(c) an insertion channel extending through each of the scope base and the outer sheath;
(d) a scope shaft actuatable relative to the scope base and slidably disposed within the insertion channel, wherein the scope shaft includes a deflectable shaft portion; and
(e) a mechanical grounding member configured to stabilize a distal portion of the outer sheath relative to a body wall of the patient, wherein the mechanical grounding member is operatively coupled to the outer sheath extracorporeally.

17. The apparatus of claim 16, wherein the mechanical grounding member includes at least one of a stabilizing arm or a stabilizing patch.

18. The apparatus of claim 16, wherein the outer sheath is configured to selectively transition between a flexible state and a rigid state.

19. A system, comprising:
(a) a support structure;
(b) a motorized drive mechanism coupled to the support structure;
(c) a surgical scope, including:
(i) a scope base coupled with the motorized drive mechanism, and
(ii) a scope shaft that is actuatable relative to the scope base by the motorized drive mechanism, wherein the scope shaft includes:
(A) a rigid proximal shaft portion, and
(B) a deflectable distal shaft portion; and
(d) a cannula, including:
(i) a proximal structure configured to be positioned extracorporeally relative to a patient,
(ii) a distal structure extending distally from the proximal structure and configured to be passed through a body wall and into a body cavity of the patient, wherein the distal structure defines a primary axis and a working channel sized and configured to receive and guide the deflectable distal shaft portion therethrough along the primary axis, and
(iii) a deflection feature configured to direct the deflectable distal shaft portion along a secondary axis that is angled relative to the primary axis.

20. The system of claim 19, further comprising a mechanical grounding member configured to stabilize a distal portion of the outer sheath relative to the body wall of the patient.

21-35. (canceled)
